(19) United States
(12) Patent Application Publication
(10) Pub. No.: US 2003/0207933 A1
(43) Pub. Date: Nov. 6, 2003

(54) Compositions and methods for increasing the sensitivity of apoptosis-resistant tumor cells to inducers of apoptosis

(76) Inventors: Yuqiang Wang, Cupertino, CA (US); Susan C. Wright, Saratoga, CA (US); James W. Larrick, Woodside, CA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/351,895

(22) Filed: Jan. 27, 2003

Related U.S. Application Data
(60) Provisional application No. 60/369,623, filed on Apr. 4, 2002.

(51) Int. Cl. A61K 31/4025; A61K 31/4015

(52) U.S. Cl. 514/422; 514/424

(57) ABSTRACT

Disclosed herein are novel compositions and methods for the treatment of cancer and other diseases. More particularly, the present invention provides compositions and methods for reversing the resistance and increasing the sensitivity of apoptosis-resistant cells to all forms of cancer therapies, such as radiation and chemotherapy, for the treatment of apoptosis-resistant cancers.

### RELATED APPLICATIONS

This application claims priority from U.S. S. No. 60/369,623, filed Apr. 4, 2002, which is incorporated herein by reference in its entirety.

### FIELD OF THE INVENTION

The present invention relates to compositions and methods for the treatment of cancer and other diseases. More particularly, the present invention relates to compositions and methods for reversing the resistance and increasing the sensitivity of apoptosis-resistant cells to all forms of cancer therapies, such as radiation and chemotherapy, for the treatment of apoptosis-resistant cancers.

### BACKGROUND OF THE INVENTION

Chemotherapy is one of the effective armamentaria in fighting cancer. However, in many cases, relapses occur because a subset of tumor cells becomes resistant to chemotherapeutic drugs. Thus, the development of drug resistance is a major problem in cancer chemotherapy. Consequently, much effort has been aimed at both understanding the mechanisms of its development and finding the means to circumvent or reverse its undesired effect.

In order for the nascent tumor to survive, it must be able to evade normal homeostatic controls as well as immune defense mechanisms. Under normal conditions, cell populations are limited by the physiological process of cell death called apoptosis (Walker, N. I., et al., Meth. Achiev. Exp. Pathol., 1988, 13:18). Evidence indicates that cells in normal tissues regulate their numbers through as yet undefined survival signals that regulate induction of apoptosis (Raff, M. C., Nature, 1992, 356:397). The ability to evade such a control mechanism confers a survival advantage to the nascent tumor cell.

The acquisition of resistance to apoptosis may be an important step in the promotion of initiated tumors (for review see Bursch, et al. TiPS, 1992, 13:245; Williams, Cell, 1991, 65:1097). Chronic exposure to certain drugs or compounds in the environment may inhibit apoptosis and act as tumor promoters (Wright, et al. FASEB J., 1993, 7:1045; Wright, et al., FASEB J., 1994, 8:654). Various genetic changes may protect a cell from apoptosis, including over-expression of oncogenes such as B cell lymphoma/leukemia-2 (Bcl-2) (Henderson, et al., Cell, 1991, 65:1107) or mutant tumor suppressor genes such as P53 (Yonish-Rouach, et al., Nature, 1991, 352:345). These genetic changes as well as other examples are associated with many types of cancer and may protect the transformed cells from host defense mechanisms as well as subvert cancer therapies (reviewed in Thompson, Nature, 1995, 267:1456).

The process of apoptosis is regulated in a positive or negative fashion by different members of the growing family of Bcl-2-related proteins (Kroemer, G., Nature Medicine, 1997, 3:614; Reed, J. C., Nature, 1997, 387:773). Overexpression of Bcl-2 is found in many hematologic malignancies, including non-Hodgkin's lymphomas (Gascoyne, R. D., et al., Blood, 1997, 90:244) and some acute myeloid leukemias (Campos, L., et al., Blood, 1993, 81:3091). Overexpression of Bcl-2 is also found in many solid tumors, including prostate cancer, breast cancer (Krajewski, S., et al., Clin. Cancer Res., 1997, 3:199; Doglioni, C., et al., Virchows Arch., 1994, 424:47), colon cancer (Bronner, M. P., et al., Am. J. Pathol., 1995, 146:20; Krajewski, M., et al., Cancer Res., 1996, 56:2422), and glioblastoma (Nakasu, S. Y., et al., Acta Neuropathol., 1994, 88:520; Alderson, L. M., et al., Cancer Res., 1995, 55:999). Furthermore, clinical studies have indicated an association between Bcl-2 expression, resistance to apoptosis, poor response to chemotherapy, and shorter patient survival (Campos, L., Blood, 1993, 81:3091; Gascoyne, R. D., Blood, 1997, 90:244). Overexpression of the Bcl-2-related anti-apoptotic protein, Bcl-X_{L}, has also been reported in a variety of malignancies (Xerri, L., et al., Br. J. Hematol., 1996, 92:900; Sclaifer, D., et al., Blood, 1995, 85:2671). Recent findings indicate that expression of Bcl-X_{L} may be an indicator of chemoresistance and is associated with a decreased response rate in multiple myeloma patients treated with a variety of chemotherapeutic drugs (Tu, Y., et al., Cancer Res., 1998, 58:256). In addition to its effects on apoptosis, there is evidence that Bcl-2 can also inhibit necrotic death (Shimizu, S., et al., Oncogene, 1996, 12:2251).

In summary, many types of cancer cells express high levels of Bcl-2 and related proteins, which protects such, cells from apoptosis induced by almost all forms of cancer therapy. A drug that circumvents the protective effects of Bcl-2 and related proteins to sensitize these cells to conventional cancer therapies could potentially extend the lives of patients or even cure drug-resistant cancer. Inhibition of different mechanisms of cell death may underlie the ability of Bcl-2 to counteract the cytotoxic effects of a wide variety of cancer treatments.

Therefore, the development of drugs that interfere with the ability of Bcl-2 and related proteins to protect cells from apoptosis may be beneficial to many cancer patients.

### SUMMARY OF THE INVENTION

The present invention provides compounds of the formula I-VII:

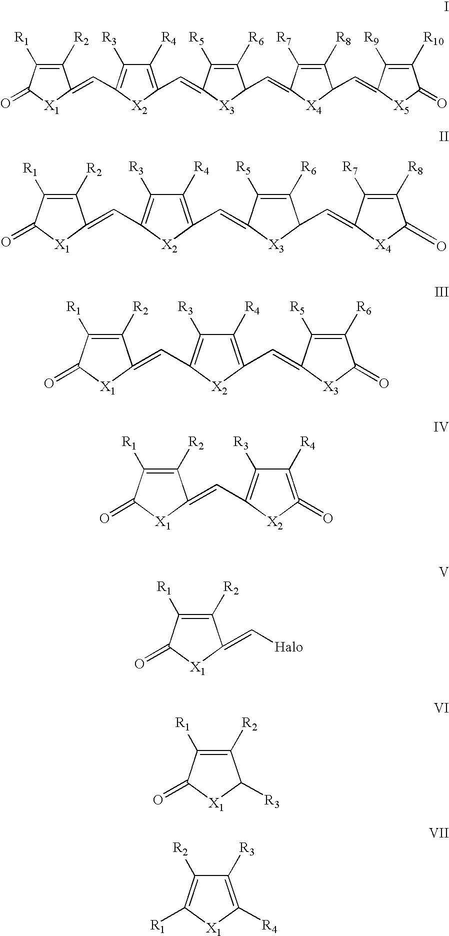

wherein: R₁, R₂, R₃, R₄, R₅, R₆, R₇, R₈, R₉, and R₁₀ are independently H, CH═CH₂, C₁-C₅ alkyl, C₁H₂COOH, C₁H₂COOCH₃, C₂H₄COOH, C₂H₄COOCH₃, C₃H₆COOH, C₃H₆COOCH₃, C₄H₈COOH, and C₄H₈COOCH₃, and wherein R₁, R₂, R₃, R₄, R₅, R₆, R₇, R₈, R₉, and R₁₀ can be the same or different; X₁, X₂, X₃, X₄, and X₅ are independently N, O or S, and wherein X₁, X₂, X₃, X₄, and X₅ can be the same or different; and Halo is F, Cl, Br or I

In a preferred embodiment, the present invention provides the following compounds:

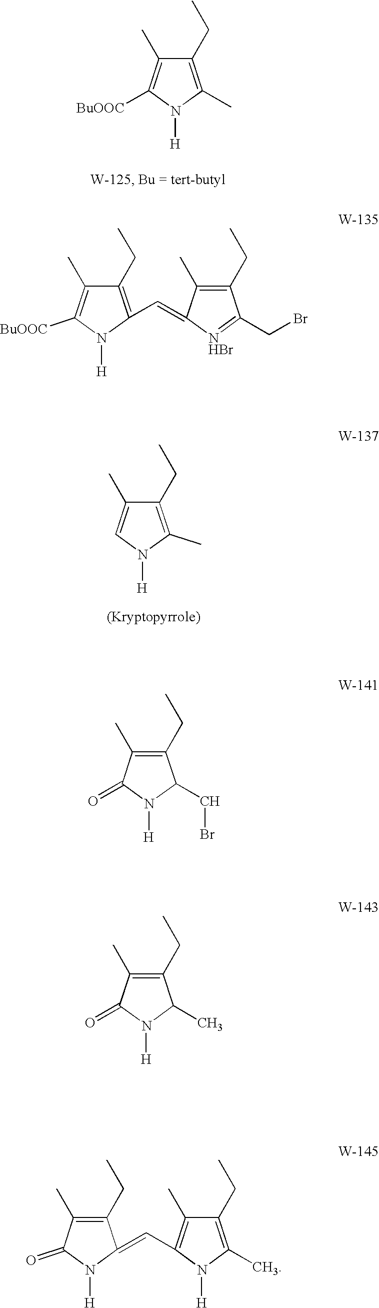

Other preferred compounds provided by the present invention include:

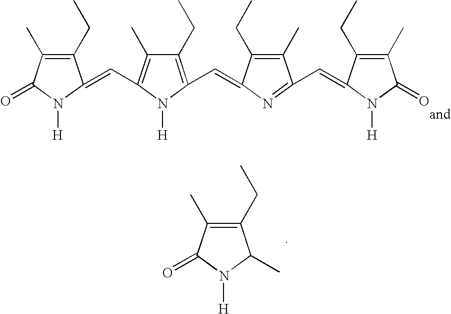

These compounds can be used as an adjuvant to conventional cancer therapy to treat apoptosis-resistant tumors and in the treatment of other diseases to overcome drug resistance.

The present invention further provides a method of reversing the resistance of an apoptosis-resistant cell. The method comprises the step of administering at least one of the following compounds having the formula:

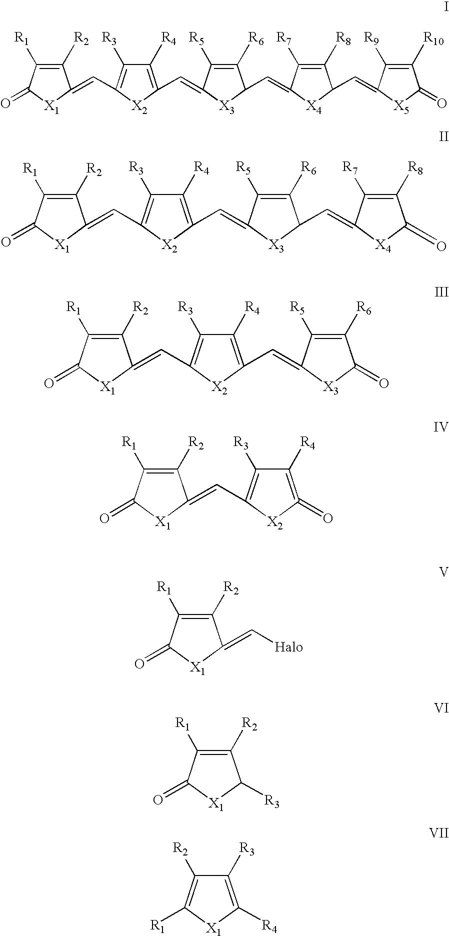

wherein: R₁, R₂, R₃, R₄, R₅, R₆, R₇, R₉, R₉, and R₁₀ are independently H, CH═CH₂, C₁-C₅ alkyl, C₁H₂COOH, C₁H₂COOCH₃, C₂ H₄COOH, C₂H₄COOCH₃, C₃H₆COOH, C₃H₆COOCH₃, C₄H₈COOH₃, and C₄H₈COOCH₃, and wherein R₁, R₂, R₃, R₄, R₅, R₆, R₇, R₈, R₉, and R₁₀ can be the same or different; X₁, X₂, X₃, X₄, and X₅ are independently N, O or S, and wherein X₁, X₂, X₃, X₄, and X₅ can be the same or different; and Halo is F, Cl, Br or I. The compound increases the sensitivity of the apoptosis-resistant cell to an apoptosis inducer.

In a preferred embodiment, the method of reversing the resistance of an apoptosis-resistant cell comprises administering at least one of the following compounds having the formula:

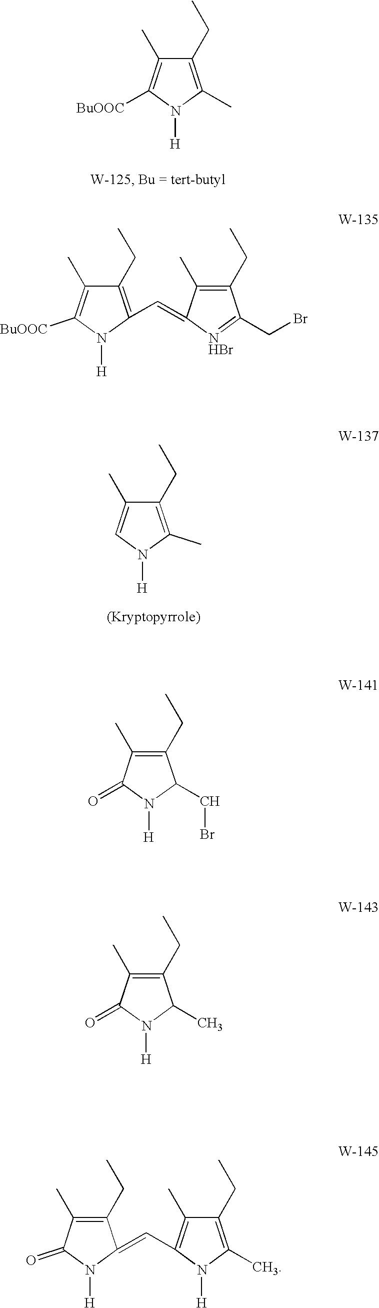

In an alternate preferred embodiment, the method of reversing the resistance of an apoptosis-resistant cell comprises administering the following compound:

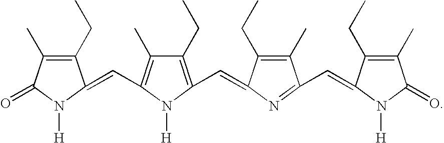

In yet another alternate preferred embodiment, the method of reversing the resistance of an apoptosis-resistant cell comprises administering the following compound:

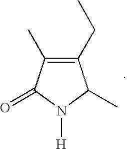

The compounds of the present invention can be administered simultaneously or sequentially with at least one apoptosis inducer. The apoptosis inducer can be a cancer therapy such as radiation therapy, chemotherapy, and biologic therapy. Preferred chemotherapy includes an antimetabolite, an alkylating agent, a plant alkaloid, and an antibiotic. Preferred antimetabolite includes methotrexate, 5-fluoruracil, 6-mercaptopurine, cytosine arabinoside, hydroxyurea, and 20chlorodeoxy adenosine. Preferred alkylating agent includes cyclophosphamide, melphalan, busulfan, cisplatin, paraplatin, chlorambucil, and nitrogen mustards. Preferred plant alkaloid includes vincristine, vinblastine, and VP-16. Preferred antibiotic includes doxorubicin, daunorubicin, mitomycin c, and bleomycin. Alternate preferred chemotherapy includes decarbazine, mAMSA, hexamethyl melamine, mitroxantrone, taxol, etoposide, dexamethasone. Preferred radiation therapy includes photodynamic therapy, radionucleotides, and radioimmunotherapy. Preferred biologic therapy includes immunotherapy, differentiating agents, and agents targeting cancer cell biology.

In a preferred embodiment, the method of the present invention is used to reverse the resistance of an apoptosis-resistant cell that overexpresses Bcl-2. In an alternate preferred embodiment, the method of the present invention is used to reverse the resistance of an apoptosis-resistant cell that overexpresses Bcl-X_{L}.

The above description sets forth rather broadly the more important features of the present invention in order that the detailed description thereof that follows may be understood, and in order that the present contributions to the art may be better appreciated. Other objects and features of the present invention will become apparent from the following detailed description considered in conjunction with the accompanying drawings. It is to be understood, however, that the drawings are designed solely for the purposes of illustration and not as a definition of the limits of the invention, for which reference should be made to the appended claims.

These and other objects of the present invention will be apparent from the detailed description of the invention provided below.

### BRIEF DESCRIPTION OF THE DRAWINGS

The present invention will be further understood from the following description with reference to the tables, in which:

FIG. 1 illustrates a model for the mechanism of apoptosis;

FIG. 2 illustrates the chemical structures of W-188 (etiobiliverdin-IV) and related analogs and derivative compounds;

FIG. 3 illustrates the synthesis of W-188 (etiobiliverdin-IV);

FIG. 4 illustrates W-188 sensitization of Bcl-2-overexpressing cells to apoptosis;

FIG. 5 illustrates W-188 sensitization of Bcl-X_{L}-overexpressing tumor cells to apoptosis induced by various chemotherapeutic drugs;

FIG. 6 depicts the augmentation by W-143 and W-188 of the cytotoxicity induced by doxorubicin (“Dox”) in Ramos B lymphoma cells that naturally express Bcl-2;

FIG. 7 illustrates that HS-treated U937 cells are sensitized by W-188 to DNA fragmentation induced by etoposide or TNF;

FIG. 8 illustrates that Wehi fibrosarcoma cells overexpressing Hsp70 are are sensitized by W-188 to DNA fragmentation induced by etoposide;

FIG. 9 illustrates that W-188 sensitizes U937 cells that resist apoptosis due to bFGF treatment;

FIG. 10 illustrates that W-188 reverses the resistance to radiation of Ly-ar cells that naturally express high levels of Bcl-2;

FIG. 11 is a chart illustrating the synergy of W-188 with doxorubicin (“Dox”) to inhibit in vivo tumor growth in the RIF-1 fibrosarcoma;

FIG. 12 illustrates the change in mouse body weight in tumor bearing animals after injection of W-188 with Dox;

FIG. 13 illustrates the augmentation by W-188 of γ-radiation-induced inhibition of HL-60-Bcl-2 xenograft growth in nude mice;

FIG. 14 illustrates the synergy of W-143 with doxorubicin (“Dox”) to inhibit tumor growth in vivo in the RIF-1 mouse model;

FIG. 15 illustrates the change in mouse body weight in tumor bearing animals after injection of W-143 with Dox; and

FIG. 16 illustrates that treatment with both W-143 and Dox completely downregulates expression of Bcl-2 in RIF-1 tumors in vivo.

### DETAILED DESCRIPTION OF THE INVENTION

Many types of cancer cells express high levels of Bcl-2 or Bcl-X_{L} which protects such cells from cell death induced by almost all forms of cancer therapy. A drug that circumvents the protective effects of Bcl-2 to sensitize these cells to conventional cancer therapies could potentially extend the lives of patients or even cure drug-resistant cancer. The present invention present invention provides such compositions and provides methods for reversing the drug-resistance and increasing the sensitivity of tumor cells to chemotherapeutic agents for the treatment of apoptosis-resistant cancers.

A. General Principles of Cancer Treatment

The goal of cancer treatment is first to eradicate the cancer. If this primary goal cannot be accomplished, the goal of cancer treatment shifts to palliation, the amelioration of symptoms, and preservation of quality of life while striving to extend life.

Cancer treatments are divided into four main groups: surgery, radiation therapy (including photodynamic therapy), chemotherapy (including hormonal therapy), and biologic therapy (including immunotherapy, differentiating agents, and agents targeting cancer cell biology). The modalities are often used in combination, and agents in one category can act by several mechanisms. For example, cancer chemotherapy agents can induce differentiation, and antibodies (a form of immunotherapy) can be used to deliver radiation therapy. Surgery and radiation therapy are considered local treatments, though their effects can influence the behavior of tumor at remote sites. Chemotherapy and biologic therapy are usually systemic treatments.

Cancer behaves in many ways as an organ that regulates its own growth. However, cancers have not set an appropriate limit on how much growth should be permitted. Normal organs and cancers share the property of having a population of cells in cycle and actively renewing and a population of cells not in cycle. In cancers, cells that are not dividing are heterogeneous; some have sustained too much genetic damage to replicate but have defects in their death pathways that permit their survival; some are starving for nutrients and oxygen; and some are reversibly out of cycle poised to be recruited back into cycle and expand if needed. Severely damaged and starving cells are unlikely to kill the patient. The problem is that the cells that are reversibly not in cycle are capable of replenishing tumor cells physically removed or damaged by radiation and chemotherapy.

Tumors follow a Gompertzian growth curve; the growth fraction of a neoplasm starts at 100% with the first transformed cell and declines exponentially over time until by the time of diagnosis at a tumor burden of 1 to 5×10⁹ tumor cells, the growth fraction is usually 1 to 4%. Cancers are actually trying to limit their own growth but are not completely successful at doing so. The peak growth rate occurs before the tumor is detectable. Metastases can be observed to grow more rapidly than the primary tumor, consistent with the idea that an inhibitory factor slows the growth of larger tumor masses. When a tumor recurs after surgery or chemotherapy, frequently its growth is accelerated and the growth fraction of the tumor is increased.

B. Principles of Chemotherapy

Historic Background

Candidate compounds that might have selectivity for cancer cells were suggested by the marrow-toxic effects of sulfur and nitrogen mustards and led, in the 1940s, to the first notable regressions of hematopoietic tumors following use of these compounds. As these compounds caused covalent modification of DNA, the structure of DNA was thereby identified as a potential target for drug design efforts. Biochemical studies demonstrating the requirement of growing tumor cells for precursors of nucleic acids led to studies of folate analogues. The cure of patients with advanced choriocarcinoma by methotrexate in the 1950s provided further impetus to define the value of chemotherapeutic agents in many different tumor types. This resulted in efforts to understand unique metabolic requirements for biosynthesis of nucelic acids and led to the design, rational for the time, of compounds that might selectively interdict DNA synthesis in proliferating cancer cells. The capacity of hormonal manipulations including oophorectomy and orchiectomy to cause regressions of breast and prostate cancers, respectively, provided a rationale for efforts to interdict various aspects of hormone function in hormone-dependent tumors. The serendipitous finding that certain poisons derived from bacteria or plants could affect normal DNA or mitotic spindle function allowed completion of the classic armamentatrium of “cancer chemotherapy agents” with proven safety and efficacy in the treatment of certain cancers.

End-Points of Drug Action

Chemotherapy agents may be used for the treatment of active, clinically apparent cancer. Table 1A lists those tumors considered curable by conventionally available chemotherapeutic agents. Most commonly, chemotherapeutic agents are used to address metastatic cancers. If a tumor is localized to a single site, serious consideration of surgery or primary radiation therapy should be given, as these treatment modalities may be curative as local treatments. Chemotherapy may be employed after the failure of these modalities to eradicate a local tumor, or as part of multimodality approaches to offer primary treatment to a clinically localized tumor. In this event, it can allow organ preservation when given with radiation, as in larynx or other upper airway sites; or sensitize tumors to radiation when given, for example, to patients concurrently receiving radiation for lung or cervix cancer (Table 1B). Chemotherapy can be administered as an adjuvant to surgery (Table 1C) or radiation, a use that may have curative potential in breast, colon, or anorectal neoplasms. In this use, chemotherapy attempts to eliminate clinically unapparent tumor that may have already disseminated. Chemotherapy can be used in conventional dose regimens. In general, these doses produce reversible acute side effects primarily consisting of transient myelosuppression with or without gastrointestinal toxicity (nausea), which are readily managed. High-dose chemotherapy regimens are predicated on the observation that the concentration-effect curve for many anticancer agents is rather steep, and increased dose can produce markedly increased therapeutic effect, although at the cost of potentially life-threatening complications that require intensive support, usually in the form of bone marrow or stem cell support from the patient (autologous) or from donors matched for histocompatibility loci (allogeneic). High-dose regimens nonetheless have definite curative potential in defined clinical settings (Table 1D).

The evaluation of a chemotherapeutic agent's benefit can be assessed by carefully quantitating its effect on tumor size and using these measurements to decide objectively the basis for further treatment of a particular patient or further clinical evaluation of a drug's potential. A partial response (PR) is defined conventionally as a decrease by at least 50% in a tumor's bi-dimensional area; a complete response (CR) connotes disappearance of all tumor; progression of disease signifies increase by >25% from baseline or best response; and “stable” disease fits into none of the above categories.

If cure is not possible, chemotherapy may be undertaken with the goal of palliating some aspect of the tumor's effect on the host. Common tumors that may be meaningfully addressed with palliative intent are listed in Table 1E. Usually tumor-related symptoms may manifest as pain, weight loss, or some local symptom related to the tumor's effect on normal structures. Patients treated with palliative intent should be aware of their diagnosis and the limitations of the proposed treatment, have access to suitable palliative strategies in the event that no treatment is elected, and have a suitable “performance status”—according to assessment algorithms such as the one developed by Kamofsky or by the Eastern Cooperative Oncology Group (ECOG). ECOG performance status 0 (PS0) patients are without symptoms; PS1 patients have mild symptoms not requiring treatment; PS2, symptoms requiring some treatment; PS3, disabling symptoms, but allowing ambulation for >50% of the day; PS4, ambulation<50% of the day. Only PS0 to PS2 patients are generally considered suitable for palliative (noncurative) treatment. If there is curative potential, even poor performance status patients may be treated, but their prognosis is usually inferior to those of good performance patients treated with similar regimens.

The usefulness of any drug is governed by the extent to which a given dose causes a useful result (therapeutic effect; in the case of anticancer agents, toxicity to tumor cells) as opposed to a toxic effect. The therapeutic index is the degree of separation between toxic and therapeutic doses. Really useful drugs have large therapeutic indices, and this usually occurs when the drug target is expressed in the disease-causing compartment as opposed to the normal compartment. Classically, selective toxicity of an agent for an organ is governed by the expression of an agent's target; or differential accumulation into or elimination from compartments where toxicity is experienced or ameliorated, respectively. Current antineoplastic agents have the unfortunate property that their targets are present in both normal and tumor tissues. Therefore, antineoplastic agents have relatively narrow therapeutic indices.

Agents with promise for the treatment of cancer have in the past been detected empirically through screening for antiproliferative effects in animal or human tumors in rodent hosts or through inhibition of tumor cells growing in tissue culture. An optimal schedule for demonstrating antitumor activity in animals is defined in further preclinical studies, as is the optimal drug formulation for a given route and schedule. Safety testing in two species on an analogous schedule of administration defines the starting dose for a phase I trial in humans, where escalating doses of the drug are given until reversible toxicity is observed. Dose-limiting toxicity (DLT) defines a dose that conveys greater toxicity than would be acceptable in routine practice, allowing definition of a maximal tolerated dose (MTD). The occurrence of toxicity is correlated if possible with plasma drug concentrations. The MTD or a dose just lower than the MTD is usually the dose suitable for phase II trials, where a fixed dose is administered to a relatively homogeneous set of patients in an effort to define whether the drug causes regression of tumors. An “active” agent conventionally has partial response rates of at least 20 to 25% with reversible non-life-threatening side effects, and it may then be suitable for study in phase III trials to assess efficacy in comparison to standard or no therapy. Response is but the most immediate indicator of drug effect. To be clinically valuable, responses must translate into effects on overall survival or at least time to progression as important indicators of an ultimately useful drug. More recently, active efforts to quantitate effects of anticancer agents on quality of life as an important outcome are being developed. Cancer drug clinical trials conventionally use a toxicity grading scale where grade I toxicities do not require treatment; grade II often require symptomatic treatment but are not life-threatening; grade III toxicities are potentially life-threatening if untreated; grade IV toxicities are actually life-threatening; and grade V toxicities ultimately lead to patient death.

Cancer arises from genetic lesions that cause an excess of cell growth or division, with inadequate cell death. In addition, failure of cellular differentiation results in altered cellular position and capacity to proliferate while cut off from normal cell regulatory signals. Normally, cells in a differentiated state are stimulated to enter the cell cycle from a quiescent state, or “G0,” or continue after completion of a prior cell division cycle in response to environmental cues including growth factor and hormonal signals. Cells progress through G1 and enter S phase after passing through “checkpoints,” which are biochemically regulated transition points, to assure that the genome is ready for replication. One important checkpoint is mediated by the p53 tumor-suppressor gene product, acting through its upregulation of the p21^{WAFI} inhibitor of cyclin-dependent kinase (CDK) function, acting on CDKs 4 or 6. These molecules can also be inhibited by the p16^{INK4A} and p27^{KIPI} CDK inhibitors and, in turn, are activated by cyclins of the D family (which appear during G1) and the proper sequence of regulatory phosphorylations. Activated CDKs 4 or 6 phosphorylate and thus inactivate the product of the retinoblastoma susceptibility gene, pRb, which in its nonphosphorylated state complexes with transcription factors of the E2F family. Phosphorylated pRb releases E2Fs, which activate genes important in completing DNA replication during S phase, progression through which is promoted by CDK2 acting in concert with cylins A and E. During G2, another checkpoint occurs, in which the cell assures the completion of correct DNA synthesis. Cells then progress into M phase under the influence of CDK1 and cyclin B. Cells may then go on to a subsequent division cycle or enter into a quiescent, differentiated state.

Biologic Basis for Cancer Chemotherapy

The classic view of how cancer chemotherapeutic agents cause regressions of tumors focused on models such as the L1210 murine leukemia system, where cancer cells grow exponentially after inoculation into the peritoneal cavity of an isogenic mouse. The interaction of drug with its biochemical target in the cancer cell was proposed to result in “unbalanced growth” that was not sustainable and therefore resulted in cell death, directly as a result of interacting with the drug's proximal target. Agents could be categorized as cell cycle-active, phase-specific (e.g., antimetabolites, purines, and pyrimidines in S phase; vinca alkaloids in M), and phase-nonspecific agents (e.g., alkylators, and antitumor antibiotics including the anthracyclines, actinomycin, and mitomycin), which can injure DNA at any phase of the cell cycle but appear to then block in G2 before cell division at a checkpoint in the cell cycle. Cells arrested at a checkpoint may repair DNA lesions. Checkpoints have been defined at the G1 to S transition, mediated by the tumor-suppressor gene p53 (giving rise to the characterization of p53 as a “guardian of the genome”); at the G2 to M transition, mediated by the chk1 kinase influencing the function of CDK1; and during M phase, to ensure the integrity of the mitotic spindle. The importance of the concept of checkpoints extends from the hypothesis that repair of chemotherapy-mediated damage can occur while cells are stopped at a checkpoint; therefore, manipulation of checkpoint function emerges as an important basis of affecting resistance to chemotherapeutic agents.

Resistance to drugs was postulated to arise either from cells not being in the appropriate phase of the cell cycle or from decreased uptake, increased efflux, metabolism of the drug, or alteration of the target, e.g., by mutation or overexpression. Indeed, the p170PGP (p170 P-glycoprotein; mdr gene product) was recognized from experiments with cells growing in tissue culture as mediating the efflux of chemotherapeutic agents in resistant cells. Certain neoplasms, particularly hematopoietic tumors, have an adverse prognosis if they express high levels of p170PGP, and modulation of this protein's function has been attempted by a variety of strategies.

Combinations of agents were proposed to afford the opportunity to affect many different targets or portions of the cell cycle at once, particularly if the toxic effects for the host of the different components of the combination were distinct. Combinations of agents were actually more effective in animal model systems than single agents, particularly if the tumor cell inoculum was high. This thinking led to the design of “combination chemotherapy” regimens, where drugs acting by different mechanisms (e.g., an alkylating agent plus an antimetabolite plus a mitotic spindle blocker) were combined. Particular combinations were chosen to emphasize drugs whose individual toxicities to the host were, if possible, distinct.

This view of cancer drug action is grossly oversimplified. Most tumors do not grow in an exponential pattern but rather follow Gompertzian kinetics, where the rate of tumor growth decreases as tumor mass increases. Thus, a tumor has quiescent, differentiated compartments; proliferating compartments; and both well-vascularized and necrotic regions. Also, cell death is itself now understood to be a closely regulated process. Necrosis refers to cell death induced, for example, by physical damage with the hallmarks of cell swelling and membrane disruption. Apoptosis, or programmed cell death, refers to a highly ordered process whereby cells respond to defined stimuli by dying, and it recapitulates the necessary cell death observed during the ontogeny of the organism. Anoikis refers to death of epithelial cells after removal from the normal milieu of substrate, particularly from cell-to-cell contact. Cancer chemotherapeutic agents can cause both necrosis and apoptosis. Apoptosis is characterized by chromatin condensation (giving rise to “apoptotic bodies”); cell shrinkage; and, in living animals, phagocytosis by surrounding stromal cells without evidence of inflammation. This process is regulated either by signal transduction systems that promote a cell's demise after a certain level of insult is achieved or in response to specific cell-surface receptors that mediate cell death signals. Modulation of apoptosis by manipulation of signal transduction pathways has emerged as a basis for understanding the actions of currently used drugs and designing new strategies to improve their use.

The current view envisions that the interaction of a chemotherapeutic drug with its target causes or is itself a signal that initiates a “cascade” of signaling steps to trigger an “execution phase” where proteases, nucleases, and endogenous regulators of the cell death pathway are activated. Effective cancer chemotherapeutic agents are efficient activators of apoptosis through signal transduction pathways. While apoptotic mechanisms are important in regulating cellular proliferation and the behavior of tumor cells in vitro, in vivo it is unclear whether all of the actions of chemotherapeutic agents to cause cell death can be attributed to apoptotic mechanisms. However, as reviewed below, changes in molecules that regulate apoptosis are clearly correlated with clinical outcomes (e.g., overexpression of Bcl-2 and related proteins).

Chemotherapeutic Agents Used for Cancer Treatment

Table 2 lists commonly used cancer chemotherapy agents and pertinent clinical aspects of their use. The drugs may be usefully grouped into three general categories: those affecting DNA, those affecting microtubules, and those acting at hormone-like receptors.

B. Mechanism of Apoptosis

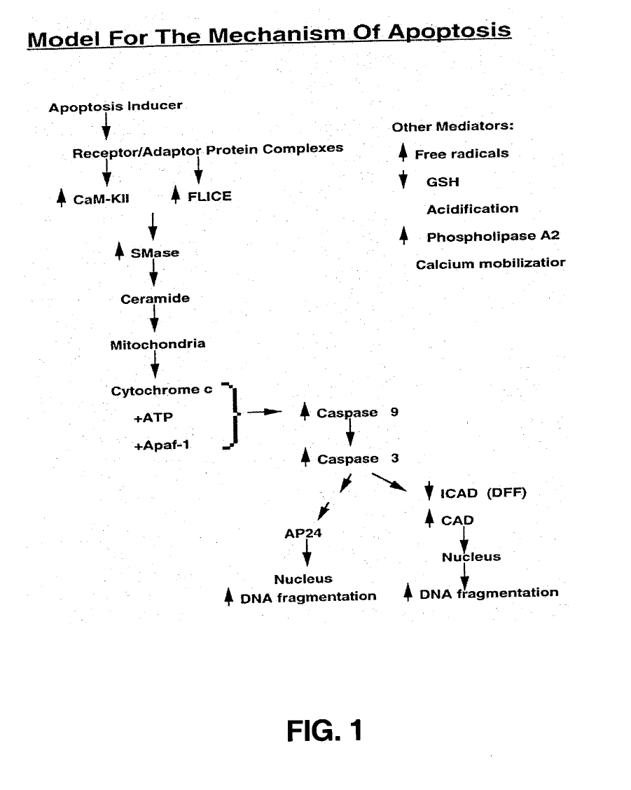

FIG. 1 provides a general working model for the mechanism of apoptosis. This model does not include all proposed mediators of apoptosis, but rather represents some of the key pathways.

Caspases consist of a family of cysteine-dependent proteases that play a key role in apoptosis (for review see Salvesen, Cell, 1997, 91:443). Activation of a protease called FLICE, or caspase 8, is one of the earliest events in TNF and Fas-induced apoptosis. Activation of calcium/calmodulin dependent protein kinase II within 3-5 minutes of exposure to either TNF or UV light has also been reported (Wright, et al., FASEB J., 1997, 11:843). These early signals may lead to the activation of sphingomyelinase (SMase), although the mechanism is not understood. SMase has been shown to transmit apoptotic signals through the production of the second messenger, ceramide (Obeid, et al., Science, 1993, 259:1769; Dressler, et al., Science, 1992, 255:1715; Wright, et al, FASEB J., 1996, 10:325). Although several actions of ceramide have been proposed, one possibility is that it contributes to mitochondrial perturbations (Susin, et al., J. Exp. Med., 1997, 186:25). Mitochondria release cytochrome c into the cytoplasm during apoptosis (Liu, et al., Cell, 1996, 86:147), which then forms a complex with ATP and Apaf-1 protein (Zou, et al., Cell, 1997, 90:405) to activate caspase 9. This enzyme proteolytically activates caspase 3, which cleaves and inactivates the recently described inhibitor of caspase-activated DNase (ICAD). This causes the release of active CAD, which digests the DNA (Enari, et al., Nature, 1998, 391:43). Caspase 3 also leads to activation of a novel serine protease called AP24 (Wright, et al., J. Exp. Med., 1994, 180:2113), which has been shown to function downstream of caspase 3 (Wright, et al., J. Exp. Med., 1997, 186:1107). In contrast to the caspases or other known proteases, AP24 has the unique ability to activate internucleosomal DNA fragmentation in isolated normal nuclei. Interestingly, inhibition of AP24 will delay or prevent apoptosis even though caspase 3 is still activated (Wright, et al., J. Exp. Med., 1997, 186:1107). AP24 is activated by many inducers of apoptosis, including chemotherapeutic drugs (Wright, et al., Biochem. Biophys. Res. Commun., 1998, 245:797). Other intracellular events that occur during apoptosis include generation of free radicals, depletion of glutathione (GSH), acidification of the cytosol, activation of phospholipase A2, and calcium mobilization. Free radical damage, as well as depletion of GSH, may be particularly relevant to the mechanism of action of Bcl-2 and related proteins as discussed below.

Bcl-2 and Cancer

The process of apoptosis is regulated in a positive or negative fashion by different members of the growing family of Bcl-2-related proteins (reviewed in Reed, Nature, 1997, 387:773; Kroemer, Nature Med., 1997, 3:199). The bcl-2 gene was originally identified at the chromosomal breakpoint of t(14;18)-bearing B-cell lymphomas. Overexpression of the 26 kDa Bcl-2 protein occurs in many different types of cancer, and may promote carcinogenesis by conferring resistance to natural defense mechanisms as well as protection from almost all forms of cancer therapies (for review see Reed, et al., J. Cell. Biochem., 1996, 60:23).

Overexpression of Bcl-2 is found in many hematologic malignancies, including non-Hodgkin's lymphomas (Gascoyne, et al., Blood, 1997, 90:244) and some acute myeloid leukemias (Campos, et al., Blood, 1993, 81:3091). Overexpression of Bcl-2 is also found in many solid tumors, including prostate cancer, breast cancer (Krajewski, et al., Clin. Cancer Res., 1997, 3:199; Doglioni, et al., Virchows Arch., 1994, 424:47), colon cancer (Bronner, et al., Am. J. Pathol., 1995, 46:20), and glioblastoma (Nakasu, et al., Acta Neuropathol., 1994, 88:520; Alderson, et al., Cancer Res., 1995, 55:999). Furthermore, clinical studies have indicated an association between Bcl-2 expression, resistance to apoptosis, poor response to chemotherapy, and shorter patient survival (Campos, et al., Blood, 1993, 81:3091; Gascoyne, et al., Blood, 1997, 90:244).

Overexpression of the Bcl-2-related anti-apoptotic protein, Bcl-X_{L}, has also been reported in a variety of malignancies (Xerri, et al., Br. J. Hematol., 1996, 92:900; Sclaifer, et al., Blood, 1995, 85:2671; Krajewski, et al., Cancer Res., 1996, 56:2422). Recent findings indicate that expression of Bcl-X_{L} may be an indicator of chemoresistance and is associated with a decreased response rate in multiple myeloma patients treated with a variety of chemotherapeutic drugs (Tu, et al., Cancer Res., 1998, 58:256).

Therefore, the development of drugs that interfere with the ability of Bcl-2 and related proteins to protect cells from apoptosis may be beneficial to many cancer patients. The rational design of such therapeutics would be facilitated by the elucidation of the as yet poorly understood mechanism of apoptosis and the means by which Bcl-2 regulates this process.

Although overexpressed Bcl-2 was originally discovered as a chromosomal translocation in follicular B cell lymphomas, it has now become clear that the Bcl-2 family members are also transcriptionally regulated. Bcl-2 (Kurland, et al., J. Biol. Chem., 2001, 276:45380), Bcl-X_{L} (Chen, et al., Mol. Cell. Biol., 2000, 20:2687; Bui, et al., J. Cell Biol., 2001, 152:753), and the Bcl-2 homologue, A1/Bfl-1 (Wang, et al., Mol. Cell. Biol., 1999, 19:5929) can be upregulated by the nuclear factor-κB (NF-κB) in various tumor cells. Inhibition of NF-κB results in the downregulation of Bcl-2 and apoptosis in EBV-transformed B lymphocytes (Feuillard, et al., Blood, 2000, 95:2068). Similar findings demonstrated protection from apoptosis in normal cells by NF-κB-induced upregulation of Bcl-X and A1 in B lymphocytes (Lee, et al., Proc. Natl. Acad. Sci. USA, 1999, 96:9136), and NF-κB-induced upregulation of A1 in macrophages (Pagliari, et al., Mol. Cell Biol., 2000, 20:8855). Activation of NFκB can promote carcinogenesis and protect malignant cells from cancer therapies by inhibiting apoptosis (reviewed in Baldwin, J. Clin. Invest., 2001, 107:241). NFκB is constitutively activated in various cancers (Rayet, Oncogene, 1999, 18:6938; Brar, et al., Am. J. Physiol. Cell Physiol., 2001, 280:C659), and is activated in response to chemotherapeutic drugs such as doxorubicin (Dox) (Bottero, et al., Cancer Res., 2001, 61:7785) and camptothecin (Chen, et al., Mol. Cell Biol., 2000, 20:2687), and ionizing radiation (Wang, et al., Science, 1996, 274:784). Therefore, there has been a great deal of interest in the development of cancer therapies that downregulate NFκB to sensitize cancer cells to drugs and radiation (reviewed in Yamamoto, J. Clin. Invest., 2001, 107:135). Besides Bcl-2, other anti-apoptotic genes that are activated by NFκB include the caspase inhibitors (c-IAP1, c-IAP-2, and XIAP), the TNF receptor-associated factors (TRAF1 and TRAF2), the Bcl-2 homolog A1, and IEX-IL (Wang, et al., Science, 1998, 281:1680; Wu, et al., Science, 1998, 281:998). Thus, an inhibitor of NFκB may promote apoptosis by affecting multiple steps in the apoptotic pathway.

The signal transducer and activator of transcription (Stat) proteins have also been shown to regulate expression of several Bcl-2 family proteins. Stats are activated after phosphorylation by the receptor-associated Janus tyrosine kinases (JAKs) and function downstream of many growth factors and cytokines (reviewed in Grad, et al., Curr. Opin. Oncol., 2000, 12:543). Activated Stats are found in a wide variety of human cancers (Catlett-Falcone, et al., Curr. Opin. Oncol., 1999, 11:490), and may function as oncogenes (Bromberg, et al., Cell, 1999, 98:295). Stat3 is constitutively activated in squamous cell carcinomas (Grandis, et al., Proc. Natl. Acad. Sci. USA, 2000, 97:4227) and U266 myeloma (Catlett-Falcone, et al., Immunity, 1999, 10:105), and downregulation of this factor decreased expression of Bcl-X_{L} and increased apoptosis. In large granular lymphocytic leukemia cells inhibition of Stat3 signalling decreased Mcl-i expression and increased apoptosis (Epling-Burnette, et al., J. Clin. Invest., 2001, 107:351). The chronic myelogenous leukemia line, K567, expresses high levels of Bcl-X_{L} due to the constitutive activation of Stat5 (Horita, et al., J. Exp. Med., 2000, 191:977).

Thus, many different types of malignancies may evade apoptosis through induction of Bcl-2 family proteins in response to activation of NF-κB or Stat transcription factors. The situation is complex in that different Bcl-2 genes may be regulated by different transcription factors in different cell types.

Mechanism of Action of Bcl-2

Bcl-2 can be found at several different intracellular locations including the mitochondrial membrane, the endoplasmic reticulum, and the nuclear membrane (Chen, et al., J. Biol. Chem., 1990, 265:4929; Jacobson, et al., Nature, 1993, 361:365), where it may modulate apoptosis by multiple mechanisms. Indeed, numerous studies provide evidence for diverse functions of Bcl-2, including inhibition of calcium mobilization (Bafy, et al., J. Biol. Chem., 1993, 268:5411; Lam, et al., Proc. Natl. Acad. Sci. USA, 1994, 91:6569), acting as an anti-oxidant to prevent free radical damage (Hockenbery, et al., Cell, 1993, 75:241; Kane, et al., Science, 1993, 262:1274), elevation of glutathione (GSH) levels (Kane, et al., Science, 1993, 2621274; Ellerby, et al., J. Neurochem., 1996, 67:1259) and/or prevention of GSH depletion (Ellerby, et al., J. Neurochem., 1996, 67:1259), and inhibition of activation of caspase 3 (Chinnaiyan, et al., J. Biol. Chem., 1996, 271:4573; Boulakia, et al., Oncogene, 1996, 12:529; Shimizu, et al., Oncogene, 1996, 12:2251), possibly secondary to its effects on mitochondria. Evidence suggests that Bcl-2 prevents mitochondria from undergoing a permeability transition, which is postulated to signal apoptotic death (Zamzami, et al., J. Exp. Med., 1996, 183:1533), as well as the release of apoptosis inducing factor (AIF) (Susin, et al., Nature, 1999, 397:441). Bcl-2 has also been shown to prevent the release from mitochondria of cytochrome c (Yang, et al., Science, 1997, 275:1129; Kluck, et al., Science, 1997, 275:1132), which is postulated to act as a cofactor in the activation of caspase 3 (Liu, et al., Cell, 1996, 86:147). Recently, overexpression of Bcl-2 was demonstrated to prevent activation of AP24 in response to TNF or UV light. This was attributed to the ability of Bcl-2 to maintain normal or elevated levels of GSH, since nutritional depletion of GSH rendered Bcl-2 overexpressing cells sensitive to activation of AP24 and apoptosis (Wright, et al., Cancer Res., 1998, 58:5570). Taken altogether, the data suggest that Bcl-2 acts at multiple sites to inhibit the apoptotic pathway, or else blocks an early signaling event that prevents the subsequent manifestations of the various apoptotic changes described above.

In addition to its effects on apoptosis, there is evidence that Bcl-2 can also inhibit necrotic death (Shimizu, et al., Oncogene, 1996, 12:2251). Inhibition of different mechanisms of cell death may underly the ability of Bcl-2 to counteract the cytotoxic effects of a wide variety of cancer treatments.

Previous Approaches to Circumvent Bcl-2-Mediated Resistance to Cancer Therapies

There has been much interest in the development of therapies that either down-regulate or inhibit the action of the anti-apoptotic Bcl-2 family members (for review see Huang, Oncogene, 2000, 19:6627). One approach is to specifically decrease the expression of the Bcl-2 protein using antisense oligonucleotides (Keith, et al., Leukemia, 1995, 9:131; Cotter, et al., Oncogene, 1994, 9:3049; Webb, et al., Lancet, 1997, 349:1137; Jansen, et al., Nature Med., 1998, 4:232) or transfection with a single-chain antibody directed against Bcl-2 (Piche, et al., Cancer Res., 1998, 58:2134). Alternatively, down-regulation of Bcl-2 by small molecules such as the nonsteroidal anti-inflammatory drug, nabumetone (Roy, et al., Br. J. Cancer, 2001, 84:1412), or the phenolic compound, carnosol (Dorrie, et al., Can. Lett., 2001, 170:33), should circumvent technical problems associated with administration of antisense oligonucleotides as drugs.

Another approach described the inactivation of Bcl-2 by phosphorylation induced by taxol or okadaic acid, which promoted apoptosis in vitro (Haldar, et al., Proc. Natl. Acad. Sci. USA, 1995, 92:4507). The tyrosine kinase inhibitor, tyrphostin AG17, induced apoptosis in a Bcl-2 overexpressing lymphoma without altering Bcl-2 protein levels in vitro (Palumbo, et al., Cancer Res., 1997, 57:2434).

Other drugs that inhibit the action of Bcl-2 without downregulation of the protein include synthetic peptides (Wang, et al., Cancer Res., 2000, 60:1498), the peripheral benzodiazepine receptor ligand, PK11195 (Fennell, et al., Br. J. Cancer, 2001, 84:1397), the microbial metabolite, Tetrocarcin A (Nakashima, et al., Cancer Res., 2000, 60:1229), and the small organic Bcl-2 ligand, HA14-1 (Wang, et al., Proc. Natl. Acad. Sci. USA, 2000, 97:7124).

Thus, at least two different approaches to therapy of Bcl-2 expressing cancers have been explored: (1) downregulation of the expression of Bcl-2 protein, and (2) blocking Bcl-2's mechanism of action without downregulation. If a single drug had both mechanisms of action, it might be a superior therapeutic.

The present invention provides compositions that can be used for reversing the drug-resistance and increasing the sensitivity of tumor cells to apoptosis inducers, such as chemotherapeutic agents and radiotherapy, for the treatment of apoptosis-resistant cancer. It is understood that the compositions of the present invention are effective for their intended purposes of enhancing sensitivity to apoptosis-inducing cancer therapies, including, but not limited to, radiation therapy (including photodynamic therapy, radionucleotides, and radioimmunotherapy), chemotherapy, and biologic therapy (including immunotherapy, differentiating agents, and agents targeting cancer cell biology). Chemotherapeutic drugs include, but are not limited to, antimetabolites, alkylating agents, plant alkaloids, and antibiotics. Antimetabolites include, but are not limited to, methotrexate, 5-fluoruracil, 6-mercaptopurine, cytosine arabinoside, hydroxyurea, and 2-chlorodeoxy adenosine. Alkylating agents include, but are not limited to, cyclophosphamide, melphalan, busulfan, cisplatin, paraplatin, chlorambucil, and nitrogen mustards. Plant alkaloids include, but are not limited to, vincristine, vinblastine, and VP-16. Antibiotics include, but are not limited to, doxorubicin (adriamycin), daunorubicin, mitomycin c, and bleomycin. Other cancer chemotherapeutic agents include DTIC (dacarbazine), mAMSA, hexamethyl melamine, mitroxantrone, taxol, etoposide, and dexamethasone.

C. Principles of Radiation Therapy

Physical Properties and Biologic Effects

Radiation therapy is a physical form of treatment that damages any tissue in its path. Tumor cells seem somewhat more sensitive to the lethal effects of radiation than normal tissues primarily because of differences in ability to repair sublethal DNA and other damage. In the target tissue, radiation damages DNA (usually single strand breaks) and generates free radicals from cell water that are capable of damaging cell membranes, proteins, and organelles. Radiation damage is dependent on oxygen; hypoxic cells are more resistant. Augmentation of oxygen is the basis for radiation sensitization. Sulfhydryl compounds interfere with free radical generation and may act as radiation protectors. The challenge for radiation treatment planning is to deliver the radiation to the tumor volume with as little normal tissue in the field as possible.

Therapeutic radiation is delivered in three ways: teletherapy with beams of radiation generated at a distance and aimed at the tumor within the patient, brachytherapy with encapsulated sources of radiation implanted directly into or adjacent to tumor tissues, and systemic therapy with radionuclides targeted in some fashion to a site of tumor. Teletherapy is the most commonly used form of radiation therapy.

Radiation from any source decreases in intensity as a function of the square of the distance from the source (inverse square law). Thus, if the radiation source is 5 cm above the skin surface and the tumor is 5 cm below the skin surface, the intensity of radiation in the tumor will be 5²/10², or 25% of the intensity at the skin. By contrast, if the radiation source is moved to 100 cm from the patient, the intensity of radiation in the tumor will be 100²/105², or 91% of the intensity at the skin. Teletherapy maintains intensity over a larger volume of target tissue by increasing the source-to-surface distance. In brachytherapy, the source-to-surface distance is small; thus, the effective treatment volume is small.

X-rays and gamma rays are the forms of radiation most commonly used to treat cancer. They are both electromagnetic, nonparticulate waves that cause the ejection of an orbital electron when absorbed. This orbital electron ejection is called ionization. X-rays are generated by linear accelerators; gamma rays are generated from decay of atomic nuclei in radioisotopes such as cobalt and radium. These waves behave biologically as packets of energy, called photons. Particulate forms of radiation are also used in certain circumstances. Electron beams have a very low tissue penetrance and are used to treat skin conditions such as mycosis fungoides. Neutron beams may be somewhat more effective than x-rays in treating salivary gland tumors. However, aside from these specialized uses, particulate forms of radiation such as neutrons, protons, and negative π mesons, which should do more tissue damage because of their higher linear energy transfer (LET) and be less dependent on oxygen, have not yet found wide applicability to cancer treatment.

A number of parameters influence the damage done to tissue by radiation. Hypoxic cells are relatively resistant. Nondividing cells are more resistant than dividing cells. In addition to these biologic parameters, physical parameters of the radiation are also crucial. The energy of the radiation determines its ability to penetrate tissue. Low energy orthovoltage beams (150 to 400 kV) scatter when they strike the body, much like light diffuses when it strikes particles in the air. Such beams result in more damage to adjacent normal tissues and less radiation delivered to the tumor. Megavoltage radiation (≧1 MeV) has very low lateral scatter; this produces a skin-sparing effect, more homogeneous distribution of the radiation energy, and greater deposit of the energy in the tumor, or target volume. The tissues that the beam passes through to get to the tumor are called the transit volume. The maximum dose in the target volume is often the cause of complications to tissues in the transit volume, and the minimum dose in the target volume influences the likelihood of tumor recurrence. Dose homogeneity in the target volume is the goal.

Radiation is quantitated on the basis of the amount of radiation absorbed in the patient, not based upon the amount of radiation generated by the machine. A rad (radiation absorbed dose) is 100 ergs of energy per gram of tissue; a gray (Gy) is 100 rad. Radiation dose is measured by placing detectors at the body surface or calculating the dose based on radiating phantoms that resemble human form and substance. Radiation dose has three determinants: total absorbed dose, number of fractions, and time. A typical course of radiation therapy should be described as 4500 cGy delivered to a particular target (e.g., mediastinum) over 5 weeks in 180-cGy fractions. Most radiation treatment programs are delivered once a day, 5 days a week in 150- to 200-cGy fractions.

The factors that influence tumor cell killing include the Do of the tumor (the dose required to deliver an average of one lethal hit to all the cells in a population), the D_{q} of the tumor (the threshold dose—a measure of the cell's ability to repair sublethal damage), hypoxia, tumor mass, growth fraction, and cell cycle time and phase (cells in late G₁ and S are more resistant). Rate of clinical response is not predictive; some cells do not die after radiation exposure until they attempt to replicate.

Compounds that incorporate into DNA and alter its stereochemistry (e.g., halogenated pyrimidines, cisplatin) augment radiation effects. Hydroxyurea, another DNA synthesis inhibitor, also potentiates radiation effects. Compounds that deplete thiols (e.g., buthionine sulfoximine) can also augment radiation effects. Hypoxia is the main factor that interferes with radiation effects.

Application to Patients

Radiation therapy can be used alone or together with chemotherapy to produce cure of localized tumors and control of the primary site of disease in tumors that have disseminated. Radiation therapy is a component of curative therapy for a number of diseases including breast cancer, Hodgkin's disease, head and neck cancer, prostate cancer, and gynecologic cancers. Radiation therapy can also palliate disease symptoms in a variety of settings: relief of bone pain from metastatic disease, control of brain metastases, reversal of cord compression and superior vena caval obstruction, shrinkage of painful masses, and opening threatened airways. In high-risk settings, radiation therapy can prevent the development of leptomeningeal disease and brain metastases in acute leukemia and lung cancer.

Brachytherapy involves placing a sealed source of radiation into or adjacent to the tumor and withdrawing the radiation source after a period of time precisely calculated to deliver a chosen dose of radiation to the tumor. This approach is often used to treat brain tumors and cervical cancer. The difficulty with brachytherapy is the short range of radiation effects (the inverse square law) and the inability to shape the radiation to fit the target volume. Normal tissue may receive substantial exposure to the radiation, with attendant radiation enteritis or cystitis in cervix cancer or brain injury in brain tumors.

Radionucleotides and Radioimmunotherapy

Nuclear medicine physicians or radiation oncologists may administer radionuclides with therapeutic effects. Iodine-131 is used to treat thyroid cancer, as iodine is naturally taken up preferentially by the thyroid. It emits gamma rays that destroy the normal thyroid as well as the tumor. Strontium-89 and samarium-153 are two radionuclides that are preferentially taken up in bone, particularly sites of new bone formation. Both are capable of controlling bone metastases and the pain associated with them, but the dose-limiting toxicity is myelosuppression.

Monoclonal antibodies and other ligands can be attached to radioisotopes by conjugation (for nonmetal isotopes) or by chelation (for metal isotopes), and the targeting moiety can result in the accumulation of the radionuclide preferentially in tumor. Iodine-131-labeled anti-CD20 and yttrium-90-labeled anti-CD20 are active in B cell lymphoma, and other labeled antibodies are being evaluated. Thyroid uptake of labeled iodine is blocked by cold iodine. Dose-limiting toxicity is myelosuppression.

Photodynamic Therapy

Some chemical structures (porphyrins, phthalocyanines) are selectively taken up by cancer cells by mechanisms not fully defined. When light, usually delivered by laser, is shone on cells containing these compounds, free radicals are generated and the cells die. Hematoporphyrins and light are being used with increasing frequency to treat skin cancer; ovarian cancer; and cancers of the lung, colon, rectum, and esophagus. Palliation of recurrent locally advanced disease can sometimes be dramatic and last many months.

D. Formulations and Methods of Treatment

Compositions intended for oral use may be prepared according to any method known to the art for the manufacture of pharmaceutical compositions and such compositions may contain one or more compounds selected from the group consisting of sweetening compounds, flavoring compounds, coloring compounds and preserving compounds in order to provide pharmaceutically elegant and palatable preparations. Tablets contain the active compound in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating compounds, for example, corn starch, or alginic acid; binding compounds, for example starch, gelatin or acacia, and lubricating compounds, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active material in admixture with excipients suitable for the manufacture of aqueous suspensions. Such excipients are suspending compounds, for example sodium carboxymethylcellulose, methylcellulose, hydropropylmethylcellulose, sodium alginate, polyvinylpyrrolidone, gum tragacanth and gum acacia; dispersing or wetting compounds may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring compounds, one or more flavoring compounds, and one or more sweetening compounds, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening compound, for example beeswax, hard paraffin or acetyl alcohol. Sweetening compounds such as those set forth above, and flavoring compounds may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting compound, suspending compound and one or more preservatives. Suitable dispersing or wetting compounds and suspending compounds are exemplified by those already mentioned above. Additional excipients, for example sweetening, flavoring and coloring compounds, may also be present.

Pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying compounds may be naturally occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation products of the said partial esters with ethylene oxide, for example sweetening, flavoring and coloring compounds, may also be present.

Syrups and elixirs may be formulated with sweetening compounds, for example glycerol, propylene glycol, sorbitol or sucrose. Such formulations may also contain a demulcent, a preservative and flavoring and coloring compounds. The pharmaceutical compositions may be in the form of a sterile injectable aqueous or oleaginous suspension. This suspension may be formulated according to the known art using those suitable dispersing or wetting compounds and suspending compounds which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The active compound may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

The active compound may be administered parenterally in a sterile medium. The drug, depending on the vehicle and concentration used can either be suspended or dissolved in the vehicle. Advantageously, adjuvants such as local anesthetics, preservatives and buffering compounds can be dissolved in the vehicle.

The compounds of the present invention are pyrrole, thiophene, and furan derivatives of the following formula:

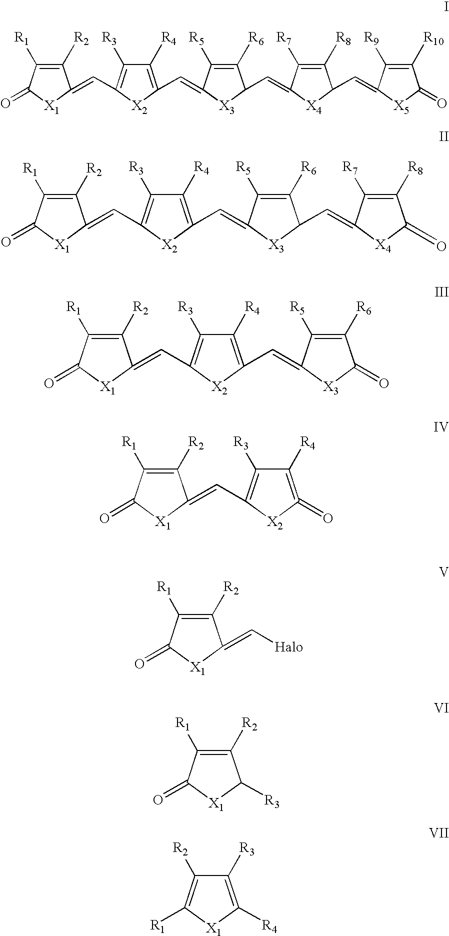

wherein: R₁, R₂, R₃, R₄, R₅, R₆, R₇, R₈, R₉, and R₁₀ are independently H, CH═CH₂, C₁-C₅ alkyl, C₁H₂COOH, C₁H₂COOCH₃, C₂H₄COOH, C₂H₄COOCH₃, C₃H₆COOH, C₃H₆COOCH₃, C₄H₈COOH, and C₄H₈COOCH₃, and wherein R₁, R₂, R₃, R₄, R₅, R₆, R₇, R₈, R₉, and R₁₀ can be the same or different; X₁, X₂, X₃, X₄, and X₅ are independently N, O or S, and wherein X₁, X₂, X₃, X₄, and X₅ can be the same or different; and Halo is F, Cl, Br or I. The compound increases the sensitivity of the apoptosis-resistant cell to an apoptosis inducer.

The present invention is also directed to novel compounds having the formulas:

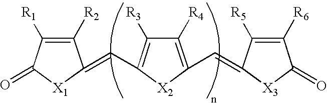

wherein: R₁, R₂, R₃, R₄, R₅, and R₆ are independently H or C₁-C₅ alkyl, and wherein R₁, R₂, R₃, R₄, R₅, and R₆ can be the same or different; X₁, X₂, and X₃ are independently NH, O or S, wherein X₁, X₂, and X₃ can be the same or different; and n=1, n=2, or n=3.

A preferred compound for use as an adjuvant to conventional cancer therapy has the structure:

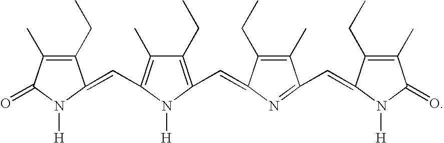

This compound is known as etiobiliverdin-IV and referred to herein as W-188. W-188 is a tetrameric structure consisting of four pyrrole rings. W-188 was first isolated with a very low yield as a byproduct in a natural product synthetic process in 1933 (J. Amer. Chem. Soc., 1933, 55:467). A high yield chemical synthesis of W-188 was reported in 1987 (Francesc, et al., J. Heterocyclic Chem., 1987, 24:1573). Few reports regarding its biological properties have been reported. This invention is based, in part, on the discovery that W-188 and structurally related analogs reverse apoptosis resistance in tumor cells overexpressing Bcl-2 and related proteins.

Another preferred compound in accordance with the present invention has the structure:

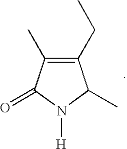

which is a monomeric derivative of W-188 and is referred to as W-143. During the initial studies with W-188 it was believed that the monomeric derivative W-143 would also have activity similar to W-188. As described herein, W-143 was shown to also sensitize 1L-60-Bcl-2 cells to etoposide-induced apoptosis. W-143 has also been shown to be water soluble and because of its size, may increase tissue penetration to improve access to tumor cells in vivo.

Compositions of the present invention (i.e., pyrrole, thiophene, and furan derivatives described above) may be administered continuously or intermittently by any route which is compatible with the particular molecules and, when included, with the particular chemotherapy agent. Thus, as appropriate, administration may be oral or parenteral, including subcutaneous, intravenous, inhalation, nasal, and intraperitoneal routes of administration. In addition, intermittent administration may be by periodic injections of a bolus of the composition once daily, once every two days, once every three days, once weekly, twice weekly, biweekly, twice monthly, and monthly.

Therapeutic compositions of the present invention may be provided to an individual by any suitable means, directly (e.g., locally, as by injection, implantation or topical administration to a tissue locus) or systemically (e.g., parenterally or orally). Where the composition is to be provided parenterally, such as by intravenous, subcutaneous, intramolecular, ophthalmic, intraperitoneal, intramuscular, buccal, rectal, vaginal, intraorbital, intracerebral, intracranial, intraspinal, intraventricular, intrathecal, intracisternal, intracapsular, intranasal or by aerosol administration, the composition preferably comprises part of an aqueous or physiologically compatible fluid suspension or solution. Thus, the carrier or vehicle is physiologically acceptable so that in addition to delivery of the desired composition to the patient, it does not otherwise adversely affect the patient's electrolyte and/or volume balance. The fluid medium for the agent thus can comprise normal physiologic saline (e.g., 0.9% aqueous NaCl, 0.15 M, pH 7-7.4). Alternatively, the use of continuous or pulsatile administration of the therapeutic compositions of the present invention by mini-pump can be employed in the methods of the present invention.

Useful solutions for parenteral administration may be prepared by any of the methods well known in the pharmaceutical art, described, for example, in REMINGTON'S PHARMACEUTICAL SCIENCES (Gennaro, A., ed.), Mack Pub., 1990. Formulations of the therapeutic agents of the invention may include, for example, polyalkylene glycols such as polyethylene glycol, oils of vegetable origin, hydrogenated naphthalenes, and the like. Formulations for direct administration, in particular, may include glycerol and other compositions of high viscosity to help maintain the agent at the desired locus. Biocompatible, preferably bioresorbable, polymers, including, for example, hyaluronic acid, collagen, tricalcium phosphate, polybutyrate, lactide, and glycolide polymers and lactide/glycolide copolymers, may be useful excipients to control the release of the agent in vivo. Other potentially useful parenteral delivery systems for these agents include ethylene-vinyl acetate copolymer particles, osmotic pumps, implantable infusion systems, and liposomes. Formulations for inhalation administration contain as excipients, for example, lactose, or may be aqueous solutions containing, for example, polyoxyethylene-9-lauryl ether, glycocholate and deoxycholate, or oily solutions for administration in the form of nasal drops, or as a gel to be applied intranasally. Formulations for parenteral administration may also include glycocholate for buccal administration, methoxysalicylate for rectal administration, or cutric acid for vaginal administration. Suppositories for rectal administration may also be prepared by mixing the therapeutic compositions of the present invention (alone or in combination with a chemotherapeutic agent) with a non-irritating excipient such as cocoa butter or other compositions that are solid at room temperature and liquid at body temperatures.

Formulations for topical administration to the skin surface may be prepared by dispersing the molecule capable of releasing the therapeutic compositions of the present invention (alone or in combination with a chemotherapeutic agent) with a dermatologically acceptable carrier such as a lotion, cream, ointment or soap. Particularly useful are carriers capable of forming a film or layer over the skin to localize application and inhibit removal. For topical, administration to internal tissue surfaces, the agent may be dispersed in a liquid tissue adhesive or other substance known to enhance adsorption to a tissue surface. For example, hydroxypropylcellulose or fibrinogen/thrombin solutions may be used to advantage. Alternatively, tissue-coating solutions, such as pectin-containing formulations may be used.

The compounds of the present invention can be used to reverse apoptosis resistance in the treatment of cancer and other diseases. The compounds of the present invention can be provideded simultaneously or sequentially in time with at least one apoptosis inducer. Where the apoptosis inducer is a composition (e.g., a chemotherapeutic drug), the compounds of the present invention can be administered in the same formulation with the apoptosis inducer or in separate formulations.

The invention is further defined by reference to the following examples, which are not meant to limit the scope of the present invention. It will be apparent to those skilled in the art that many modifications, both to the materials and methods, may be practiced without departing from the purpose and interest of the invention.

### EXAMPLES

### Example 1

### Synthesis of W-143 and W-188

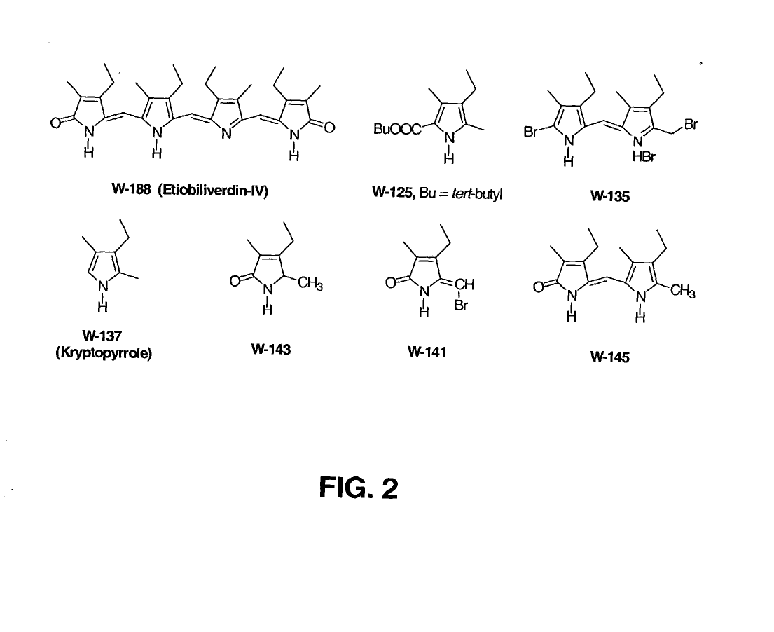

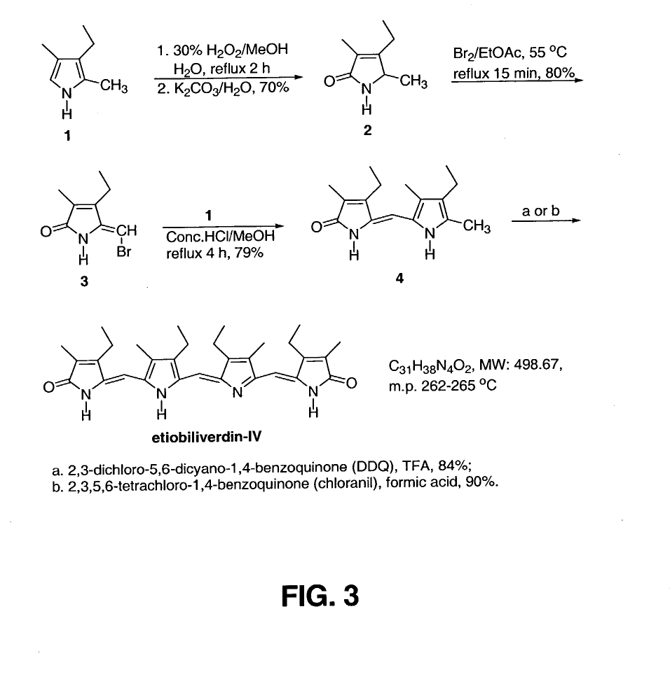

FIG. 2 illustrates the chemical structures of W-188 (etiobiliverdin-IV) and related analogs and derivative compounds. FIG. 3 illustrates the synthesis of W-143 and W-188 (etiobiliverdin-IV), as described below.

3,5-dimethyl-4-ethyl-2-oxo-2,5-dihydropyrrole (2) (W-143)

Kryptopyrrole, 1, (380 g, 3.1 mol) in MeOH (1 L) and water (400 ml) purged with nitrogen in a round bottom flask is heated to 50° C. while maintaining a slight flow of nitrogen. Hydrogen peroxide (30%, 380 ml, 3.35 mol) is added slowly such that the reaction temperature does not exceed 60° C. After all of the hydrogen peroxide is added, the reaction mixture is stirred at 50° C. for another 2 h, and then refluxed for 2 h. After cooling, a solution of K₂CO₃ (80 g) in water (180 mL) is added and the mixture is stirred overnight. Water (1 L) is added and the mixture is extracted with dichloromethane. The aqueous layer is neutralized with diluted HCl and again extracted with dichloromethane. The combined organic solution is washed with brine and dried using sodium sulfate. Solvent is removed and the product is purified using column chromatography on silica gel to afford white crystals after crystallization, (70% yield), m.p. 83° C.

5-Bromomethylene-4-ethyl-3-methyl-2-oxo-2,5-dihydropyrrole (3)

To 2 (135.5 g, 0.97 mol) in anhydrous ethyl acetate (1.5 L) is added bromine (310 g, 1.94 mol) slowly such that the reaction temperature does not exceed 55° C. After the addition is completed, the reaction mixture is refluxed for 15 min. The ethyl acetate phase is washed with 5% NaHCO₃ (3×150 mL) and then with 10% NaHCO₃ (3×150 mL). The organic solution is dried using sodium sulfate. The product is crystallized after solvent is removed to afford 168 g of yellow needles (80% yield), m.p. 140-142° C.

Kryptopyrromethenone (KRP) (4)

To a solution of 1 (1.45 g, 11.5 mmol) and 3 (2.5 g, 11.5 mmol) in methanol (100 mL) is added concentrated HCl (3 drops) under nitrogen. The reaction mixture is heated to reflux for 4 h then allowed to cool. The yellow-orange precipitate is filtrated and recrystallized in benzene to afford 2.25 g of yellow needles (79% yield), m.p. 250-251° C.

W-188 (etiobiliverdin-IV)

To 4 (129 mg, 0.5 mmol) in dry THF (50 mL) containing TFA (2.5 ML) is added 2,3-dichloro-5,6-dicyano-1,4-benzoquinone (DDQ, 136 mg, 0.6 mmol) dissolved in THF (25 mL) dropwise over a period of 90 min while maintaining the temperature at 20° C. The mixture is cooled in an ice bath for 5 min and diluted with an ice-cold mixture of chloroform (50 ml) and 1% triethylamine in water (75 mL). The aqueous phase is washed with chloroform (2×15 mL) and the combined organic phase is washed with 0.1 M NaHCO₃ solution until the aqueous phase shows a neutral pH. Solvent is removed and the product is purified using column chromatography on silica gel eluting with chloroform and methanol (50:1) to afford 104 mg (84% yield) of dark solid, m.p. 262-265° C.

### Example 2

### Evaluation of the Bcl-2 Sensitizing Activity of W-188 and Structurally-Related Analogs and Derivatives

Evaluation of the Bcl-2 sensitizing activity of W-188

The present study evaluated the ability of W-188 to sensitize Bcl-2-overexpressing cells to induce apoptosis. The human myelogenous leukemia, HL-60, and the human B cell leukemia, PW, normally express very low levels of Bcl-2. As a model system, W-188 and various analogs and derivatives were tested in normal HL-60 and PW cells and in Bcl-2-transfected HL-60 and PW cells that overexpress high levels of Bcl-2 (see Gilbert et al., J. Cell Physiol., 1996, 171:299).

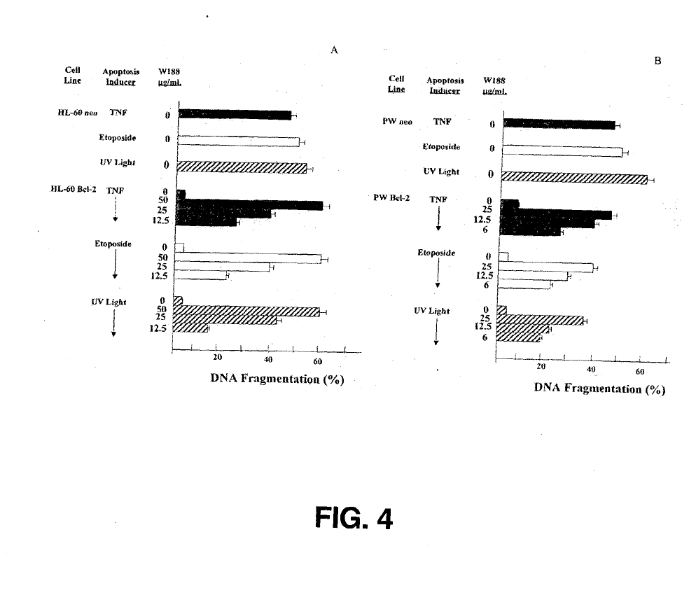

As depicted in FIG. 4, HL-60 cells (Panel A) and PW cells (Panel B) cells were exposed to various apoptosis inducers: TNF at 5 ng/ml; etoposide at 20 μM; and UV at 0.08 J/cm². The release of 3H-labeled DNA fragments was measured after 5 h. Normal HL-60 and PW cells were equally sensitive to apoptosis inducers as compared with cells that were transfected with vector encoding neomycin resistance (HL-60 neo and PW neo) only. The results shown in FIG. 4A demonstrate that HL-60 Bcl-2 cells are highly resistant to DNA fragmentation induced by tumor necrosis factor (TNF), etoposide, and UV light as compared with HL-60 neo. However, the addition of W-188, dose-dependently sensitized HL-60 Bcl-2 cells to DNA fragmentation induced by all three agents. The optimal doses of 25-50 μg/mL of W-188 caused HL-60 Bcl-2 cells to undergo DNA fragmentation at levels comparable with those seen with HL-60 neo cells. Similar results were obtained with PW-Bcl-2 cells as shown in FIG. 4B. W-188 alone was nontoxic. The addition of W-188 to the neo cell lines caused a 1.5-2 fold increase in DNA fragmentation in response to the various apoptosis inducers.

Evaluation of the Bcl-X_{L} sensitizing activity of W-188

The above studies show that W-188 enhances the cytotoxic effects of apoptosis inducers on tumor cell lines overexpressing Bcl-2. Since Bcl-X_{L} has also been implicated in resistance to cancer therapies, the present study evaluated the ability of W-188 to counteract the protective effects of this Bcl-2 family member.

It has been reported that the murine myeloma, SP2/0, is highly sensitive to cycloheximide-induced apoptosis, whereas the related murine myeloma, P3X63Ag8.653 (P3X), was resistant (Gauthier, E. R., et al., Cancer Res., 1996, 56:1451). This resistance was attributed to natural high levels of expression of Bcl-X_{L} in the absence of any detectable Bcl-2. In contrast, SP2 expressed neither Bcl-2 or Bcl-X_{L}. Thus, normal (SP2) or Bcl-X_{L}-overexpressing cells (P3X) were treated with chemotherapeutic drugs ±0.1 μg/ml W-188. After 24 h, % DNA fragmentation was measured by release of ³H-labeled DNA fragments.

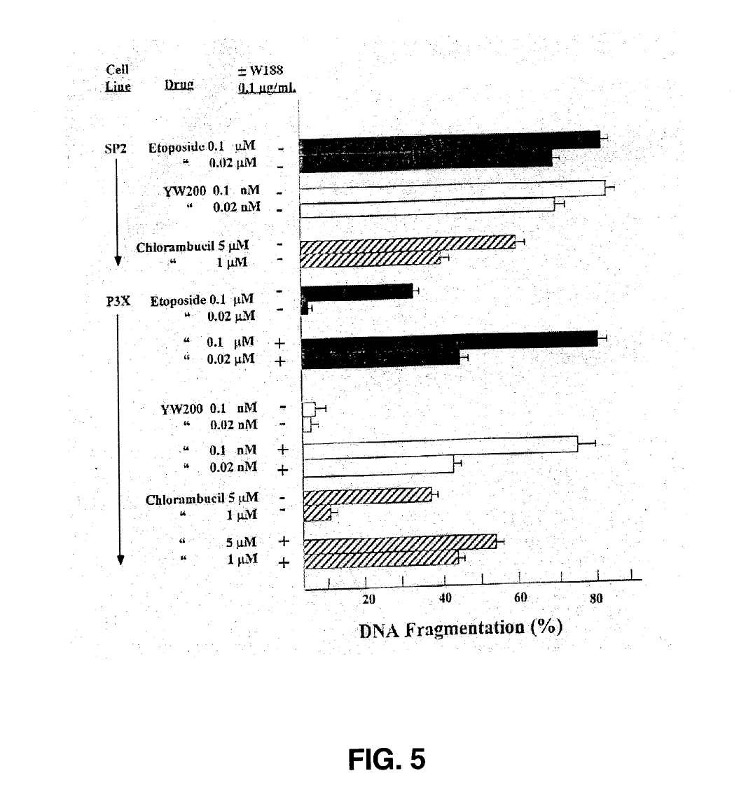

P3X was relatively resistant to DNA fragmentation induced by three chemotherapeutic drugs, etoposide, chlorambucil, and YW-200, relative to SP2 (FIG. 5). YW-200 is an experimental DNA-binding chemotherapeutic drug related to CC-1065 (Wang, Y., et al., U.S. Pat. No. 5,843,937, 1998). The addition of W-188 at only 0.1 μg/mL sensitized P3X to undergo drug-induced DNA fragmentation at levels close to or equal to those of SP2.

These results suggest that W-188 also enhances the cytotoxic effects of apoptosis inducers on tumor cell lines overexpressing Bcl-X_{L}.

Evaluation of the Sensitizing Activity of W-188 in Normal Non-Transformed Cells

The above studies show that W-188 enhances the cytotoxic effects of apoptosis inducers on three tumor cell lines overexpressing Bcl-2 or Bcl-X_{L}. However, for W-188 to be a useful adjuvant to conventional cancer therapies, it is desirable that it does not enhance drug-induced cytotoxicity of normal non-transformed cells. Therefore, the effects of W-188 on drug-induced killing of normal venous endothelial cells were tested.

The normal venous endothelial cell line does not undergo apoptosis, but instead dies by necrosis on exposure to chemotherapeutic drugs. The results shown in Table 3 demonstrate that W-188 at concentrations as high as 50 μg/mL did not augment the killing of endothelial cells by etoposide or YW-200.

This finding suggests that W-188 does not increase the toxic side effects of chemotherapy to normal host tissues.

Evaluation of the Sensitizing Activity of W-188 Analogs and Derivatives

Although the mechanism underlying the sensitizing activity of W-188 in tumor cells overexpressing Bcl-2 and related proteins is not currently known, the action of W-188 is unique.

In the present study, a series of compounds structurally related to W-188 (W-125, W-135, W-137, W-141, W-143 and W-145, illustrated in FIG. 2) was synthesized and tested for sensitizing activity in tumor cells overexpressing Bcl-2. W-188 was the most potent among the compounds tested. However, the compounds structurally related to W-188 also sensitized Bcl-2-overexpressing tumor cells to chemotherapeutic agent, as shown in Table 4. Although W-188 was the most potent compound, other compounds may have certain characteristics that make them attractive drug candidates. For example, W-143 is water soluble whereas W-188 is not, and the smaller size of W-143 may increase tissue penetration and improve access to tumor cells in vivo.

Evaluation of W-188 and W-143 in Cell Lines that Naturally Express Bcl-2

In the present study, the sensitizing activity of W-188 and W-143 was tested in the Raji and Ramos cell lines. Since these two human B lymphoma lines naturally express high levels of anti-apoptotic Bcl-2 proteins, they may be more clinically relevant than the transfected HL-60-Bcl-2 line.

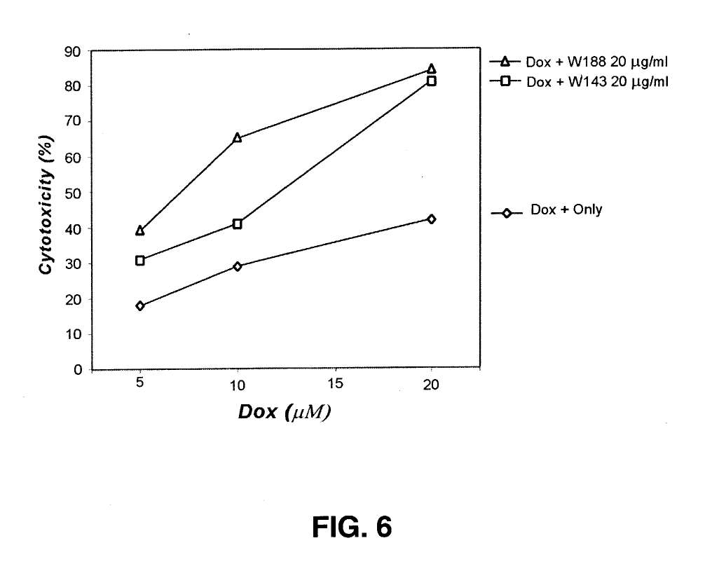

The sensitizing effects of W-143 and W-188 were first tested on these lines in vitro. Preliminary experiments with these cells revealed that chemotherapeutic drug-induced cell death occurred through both apoptosis and necrosis. In order to be able to detect both forms of death, cell death was measured by trypan blue exclusion, as opposed to DNA fragmentation, which does not measure necrosis. Ramos cells were cultured with different concentrations of Dox with and without W-143 or W-188 at 20 μg/ml for 24 hr, and then viability assessed microscopically by counting at least 100 cells. W-143 or W-188 treatment alone was completely nontoxic (data not shown). The results shown in FIG. 6 demonstrate that both W-143 and W-188 increased the cytotoxicity mediated by Dox over two fold. Similar results were seen with the Raji cell line (data not shown). Therefore, these data suggest that these cell lines seem appropriate to test W-143 and W-188 in the xenograft model.

### Example 3

### W-188 Sensitizes Bcl-2-Overexpressing Cells to Etoposide-Induced Activation of AP24

Numerous studies indicate that Bcl-2 may act at different points in the apoptotic pathway to prevent cell death. The present inventors have published results indicating that overexpression of Bcl-2 attenuates activation of the AP24 serine protease in response to apoptosis-inducers (Wright, S. C., et al., Cancer Res., 1998, 58:5570). Furthermore, nuclei isolated from Bcl-2-overexpressing cells were relatively resistant to DNA fragmentation induced by AP24 in a cell-free assay (Wright, S. C., et al., Cancer Res., 1998, 58:5570). Therefore, the effects of W-188 in this system were tested in the present study.

HL-60-neo and PW-neo cells exposed to apoptosis inducers showed approximately a ten fold increase in AP24 activity isolated from the cytosol of cells prior to cell death (Table 5). In contrast, HL-60-Bcl-2 and PW-Bcl-2 did not activate AP24 unless they were also treated with W-188. Nuclei isolated from HL-60-Bcl-2 cells were resistant to AP24-induced DNA fragmentation, as expected (Wright, S. C., et al., Cancer Res., 1998, 58:5570). However, pretreatment of HL-60-Bcl-2 cells with W-188 restored the sensitivity of the isolated nuclei to AP24-induced DNA fragmentation (Table 6).

These findings support the hypothesis that Bcl-2 acts on at least two events that are both attenuated by W-188: (1) signal transduction leading to activation of AP24; and (2) induction of nuclear DNA fragmentation by activated AP24. The observation that Bcl-2 can protect nuclei from apoptotic changes is consistent with the fact that Bcl-2 is found in the nuclear membrane (Chen, Z., et al., J. Biol. Chem., 1990, 265:4929). It has also been reported that Bcl-2 can protect isolated nuclei from apoptotic changes induced by granzyme B (Voehringer, D. W., et al., Proc. Natl. Acad. Sci. USA, 1998, 95:2956), providing further support for the concept that Bcl-2 can attenuate multiple apoptotic signals, including nuclear events.

### Example 4

### W-188 Sensitizes Cells Resistant to Apoptosis Through Non-Bcl-2 Mechanisms

It is now clear that malignant cells may employ many different mechanisms to evade death induced by host defenses or cancer therapies (for review see White, M. K. Leukemia, 2001, 15:1011). The present study assessed whether the compositions of the present invention could sensitize tumor cells that resist apoptosis through mechanisms other than Bcl-2.

Apoptosis Resistance Induced by Overexpression of Heat Shock Proteins (HSPs)

Overexpression of heat shock proteins (HSPs) is commonly found in many types of cancers (Jaattela, M. Ann. Med., 1999, 31:261), and these proteins confer resistance to apoptosis induced by chemotherapeutic agents (Samali, A., Exp. Cell Res., 1996, 223:163).

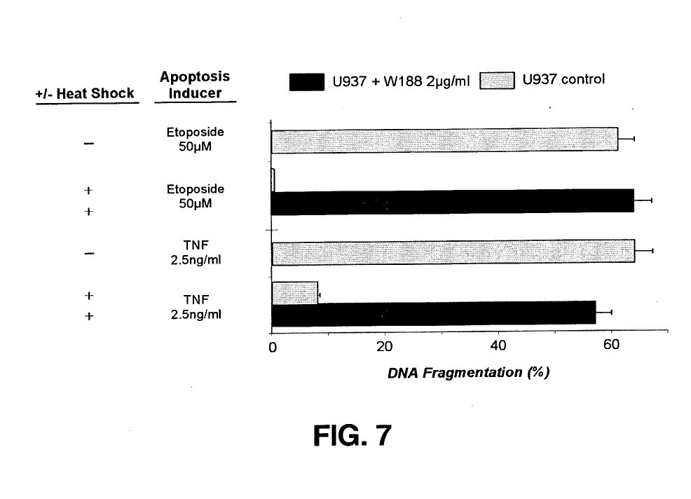

U937 cells received either no heat shock (HS) treatment or HS treatment at 42° C. for 30 min followed by recovery for 3 hr. The cells were then treated with etoposide or TNF with and without W-188, which alone was nontoxic. After 3 hr, DNA fragmentation was measured by release of ³H-labeled DNA. As expected, U937 cells exposed to HS treatment were completely resistant to etoposide or TNF-induced DNA fragmentation (FIG. 7). However, exposure of cells to W-188 following HS treatment resulted in levels of etoposide or TNF-induced DNA fragmentation comparable to that seen in normal U937 cells. These results suggest that the compositions of the present invention are able to sensitize tumor cells that resist apoptosis through overexpression of HSPs.

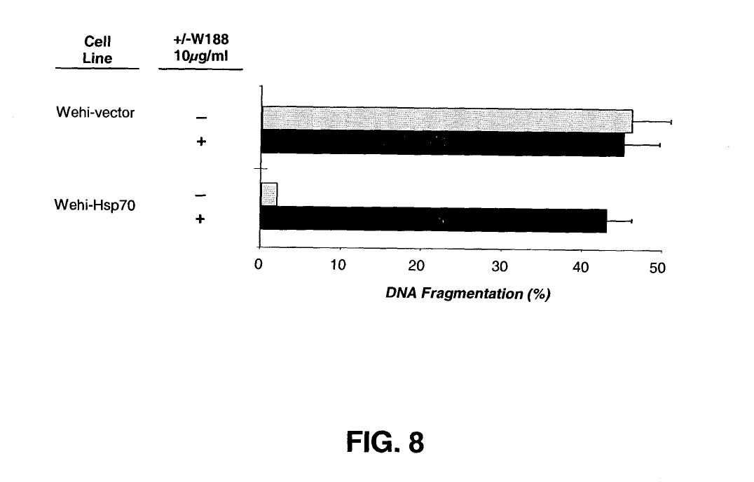

This finding was confirmed using the Wehi murine fibrosarcoma cell line that is transfected with and overexpresses Hsp70, as described previously (Jaattela, M., et al., EMBO J. 1998, 17:6124). Wehi transfected with Hsp70 or vector control were cultured with etoposide ±20 μM W-188 in a 20 h DNA fragmentation assay. Wehi-Hsp70 exposed to both W-188 and etoposide underwent DNA fragmentation at levels comparable to that observed with the Wehi-vector control (FIG. 8). Similar results were obtained when apoptosis was assessed by the morphologic changes characteristic of apoptosis in these cells (cell rounding, detachment and membrane blebbing) (not shown).

Apoptosis Resistance Induced by Growth Factors

Another means by which cells may evade apoptosis is by treatment with various growth factors. To assess the ability of W-188 to reverse the apoptosis resistance induced by growth factors, U937 cells were treated ±1 ng/ml basic fibroblast growth factor (bFGF) for 20 h, then exposed to etoposide or TNF and DNA fragmentation measured.

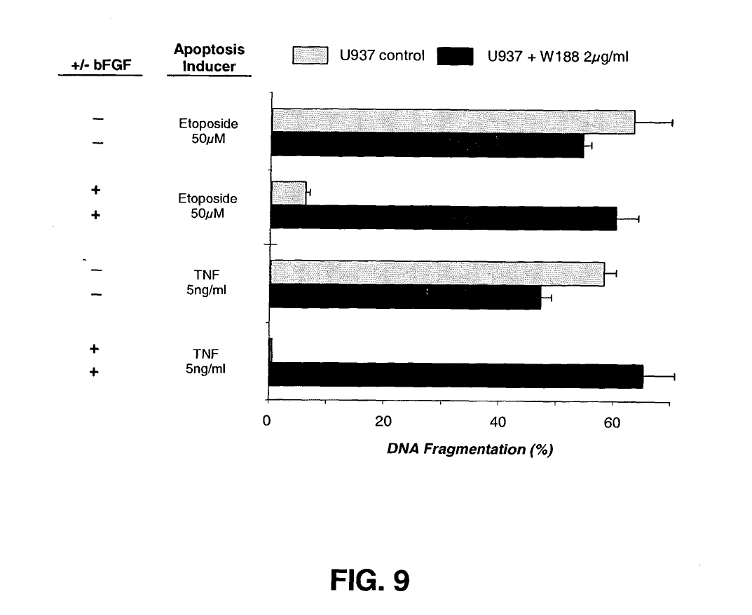

The treatment of U937 cells with basic fibroblast growth factor (bFGF) for 20 h resulted in complete resistance to etoposide or TNF-induced DNA fragmentation (FIG. 9). However, as depicted in FIG. 9, the addition of W-188 following bFGF pretreatment completely reversed resistance to apoptosis.

These results suggest that W-188 and related analogs and derivatives may alter a cell's susceptibility to apoptosis by affecting responses that are independent of Bcl-2.

### Example 5

### W-188 Reverses Resistance to Radiation by LY-ar Cells that Naturally Express High Levels of Bcl-2

The present study demonstrated that W-188 can promote apoptotic signal transduction events even in the presence of enforced overexpression of exogenous Bcl-2. Additional studies were performed to determine if W-188 could also sensitize cells that naturally express high levels of Bcl-2. Clones were isolated from the murine B cell lymphoma, LY, that naturally express low levels of Bcl-2 (LY-as) or high levels (LY-ar), as described previously (Mirkovic, N., et al., Oncogene, 1997, 15:1461).

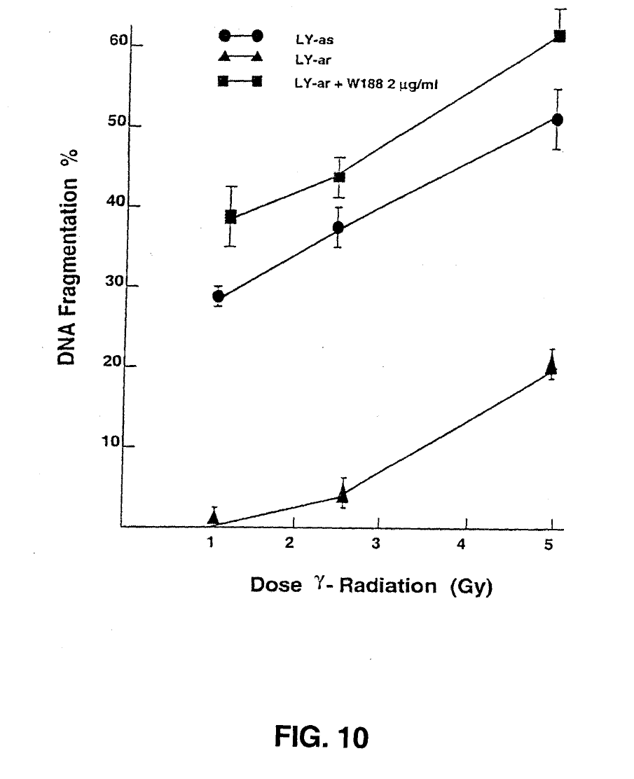

LY-ar was highly resistant to apoptosis induced by y-radiation. However, in the presence of W-188, LY-ar cells underwent DNA fragmentation at levels equal to or exceeding that of LY-as treated with radiation (FIG. 10). W-188 also sensitized HL-60-Bcl-2 and LY-ar cells to cell death mediated by etoposide and chlorambucil as measured in a 10 day colony formation assay (not shown). In contrast, W-188 did not increase the toxic effects of etoposide on normal human venous endothelial cells or normal rat intestinal epithelial cells. The apparent selective effects of W-188 may be due to the preferential intracellular accumulation of porphyrin compounds in transformed cells as opposed to normal cells reported previously (Bugelski, P. J., et al., Cancer Res., 1981, 41:4606).

W-188 was then compared to other compounds previously reported to counteract Bcl-2 (Marchetti, P., et al., J. Immunol., 1996, 157:4830; Hirsch, T., et al., Exp. Cell Res., 1998, 241:426). W-188 at 2.5 [M completely reversed the effect of overexpression of Bcl-2, whereas protoporphyrin IX and the peripheral benzodiazipine ligand, PK11195, both at 20 μM (the highest non-toxic concentration) were completely ineffective (data not shown). Furthermore, taxol, also reported to inactivate Bcl-2 (Haldar, S., et al., Cancer Res., 1996, 56:1253), was also inferior to W-188 in the present study (data not shown).

Experiments to study the mechanism of action revealed that treatment with W-188 alone did not affect intracellular levels of GSH or caspase 3 activity (DEVDase). However, combined treatment with W-188 and etoposide caused depletion of GSH and activation of caspase 3 in HL-60-Bcl-2 and LY-ar cells at levels comparable to that seen in HL-60-neo and LY-as cells. In contrast, treatment with W-188 and etoposide did not downregulate the expression of Bcl-2 protein in HL-60-Bcl-2 cells. These findings suggest that W-188 can promote apoptotic signal transduction events even in the presence of enforced overexpression of exogenous Bcl-2. However, more recent findings indicate that W-188 may also downregulate expression of endogenous Bcl-2 in cells that naturally have high levels of this protein.

### Example 6

### Subcellular Distribution of W-188

To further investigate the mechanism of action of W-188, the subcellular distribution of W-188 was assessed in the present study.

A method to measure the subcellular localization of this compound was developed by the inventors of the present invention. Because of its distinctive absorbance spectrum, W-188 can be detected in cell and tissue extracts by measuring absorbance at 380 nm, and quantitated relative to a standard curve constructed with known amounts of W-188. HL-60 and PW cells containing vector control or overexpressing Bcl-2 were incubated with W-188 followed by cell lysis and isolation of nuclei, mitochondria, and cytotol by differential centrifugation.

Measurement of W-188 in the different compartments revealed that most of the W-188 accumulated in the nucleus and mitochondria, with little or none detected in the cytosol (Table 7). Furthermore, it can be seen that nuclei and mitochondria from cells overexpressing Bcl-2 accumulated higher levels of W-188 than the vector control cells. These findings are consistent with the hypothesis that W-188 may counteract the protective effects of Bcl-2 mediated at both the mitochondria as well as the nucleus.

### Example 7

### In Vivo Toxicity of W-188 and W-143 in Mice

A. Acute Single Dose Toxicity

The present study assessed the acute single-dose toxicity of W-188 and W-143.

Solutions of W-188 were dissolved in DMSO and injected i.p. in groups of three mice each as well as vehicle control at 500 mg/kg. No deaths were observed, and both vehicle control and W-188 injected mice gained weight normally over the 1.5-month observation period.

Since W-143 is water-soluble, higher doses could be tested. All three mice injected with 1000 mg/kg of W-143 died within 24 h. However, a dose of 500 mg/kg did not cause death, and all mice gained weight normally over a three-week observation period, increasing from 22 g to 27 g in both vehicle and drug-treated groups.

Therefore, the MTD (maximum tolerated dose) for a single dose of either compound is estimated to be 500 mg/kg.

B. Multiple Dose Toxicity Simulating Conditions for RIF-] Tumor Model

In the present study, more extensive toxicity studies with histopathological analysis of seven different tissues was performed on W-143 at a commercial lab.

Groups of six mice (3 male and 3 females) each were given three i.p. injections at two day intervals of vehicle, doxorubicin 5 mg/kg alone, W-143 200 mg/kg alone or both together. On day 5 and day 15 after the last injection, mice were sacrificed, necropsied, and tissue samples processed. Tissues examined include bone marrow, liver, heart, small and large intestine, 1 sex organ, and kidney. Drug-treated mice sacrificed on day 5 had minor vacuolar changes in the liver (Dox alone, W-143 alone, or both combined), while the rest of the tissues were normal. Mice sacrificed on day 15 had normal necropsies and all tissues were normal, indicating that they recovered from the drug effects observed on day 5.

These results suggest that W-143, when administered with a chemotherapeutic drug, will not cause unacceptable toxic side effects.

### Example 8

### W-188 AND W-143 Synergizes with Dox to inhibit RIF-1 Tumor Growth In Vivo in Mice

The present study tested in the ability of W-188 to synergize with Dox and inhibit tumor growth in vivo. The RIF-1 fibrosarcoma model was used because this model has been well-characterized, and naturally expresses high Bcl-2 levels and has a consistent growth pattern (Nahabedian, M. Y., et al., J. Natl. Can. Inst., 1988, 80:739; Chang, M-J., et al., Cancer Res., 1994, 54:5380; Srivastana, M., et al., J. Biol. Chem., 2001, 276:15481).

Groups of five mice each were inoculated s.c. with RIF-1 cells. The tumors were allowed to grow for 12 days to a size of 0.5 cm diameter, which was designated day 0. The mice were then given five i.p. injections every other day with vehicle, Dox 2.5 mg/kg, Dox 5.0 mg/kg, W-188 100 mg/kg, and Dox 5.0 mg/kg+W-188 100 mg/kg. The mice were weighed and the tumor size measured every day.

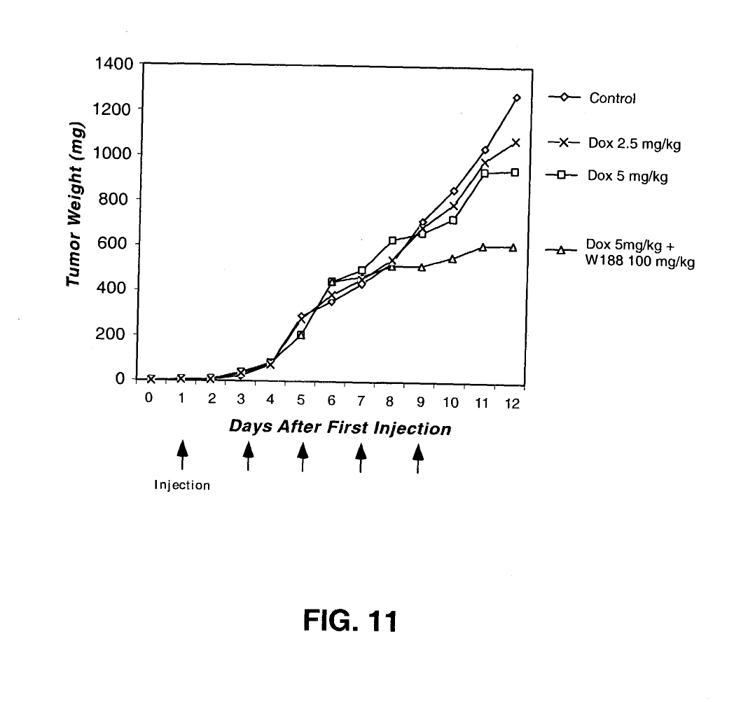

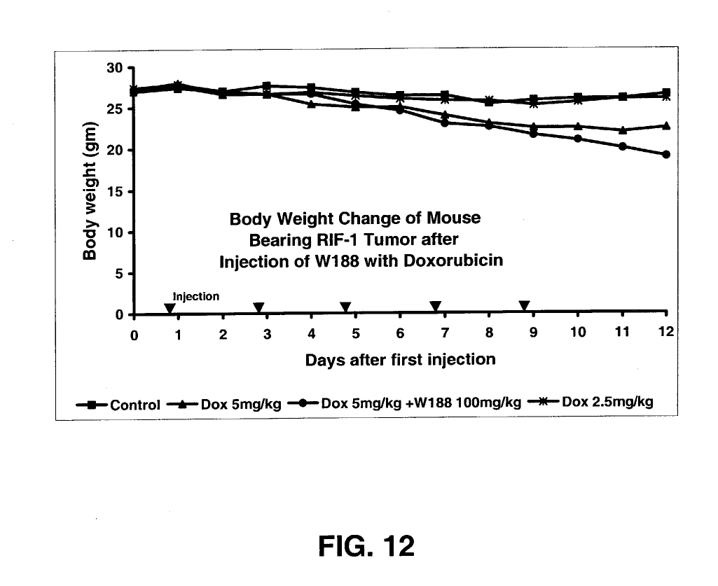

The results show that treatment with Dox alone at 2.5 or 5 mg/kg had very little effect on tumor growth (FIG. 11). Treatment with W-188 alone had no effect on tumor growth (data not shown). However, treatment with both W-188 and Dox 5.0 mg/kg synergistically inhibited tumor growth. On day 12, the tumors of mice treated with the combination of drugs were only half the size of the vehicle control (p=0.0005). Mice treated with W-188 alone did not show any decrease in weight compared to the vehicle control (data not shown). Mice treated with both W-188 and Dox 5 mg/kg did lose weight, but not much more than those mice treated with Dox 5 mg/kg alone (FIG. 12).

### Example 9

### W-188 Augments γ-Radiation Inhibition of HL-60 Bcl-2 Xenograft Growth in Mice

This study tested in the ability of W-188 to augment the γ-radiation inhibition of tumor growth in vivo in mice.

Groups of five nude mice were injected s.c. with 5×10⁶ HL-60-Bcl-2 cells, and tumors allowed to grow to a size of 7-10 mm. Tumors were then exposed to a single dose of γ-radiation, a single dose of W-188 or vehicle control, or a single dose of both γ-radiation and W-188. Tumor growth was monitored every other day, and when the tumor volume increased four fold, the animals were sacrificed.

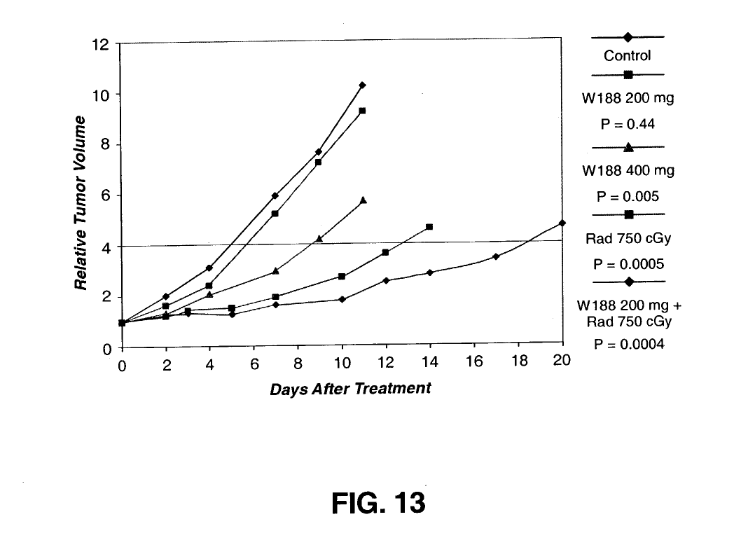

The results in FIG. 13 show that treatment with 400 mg/kg W-188 alone significantly inhibited tumor growth compared to the vehicle control (p=0.005). However, treatment with W-188 200 mg/kg had no significant effect. Treatment with radiation alone extended tumor re-growth time, however combined treatment with radiation and W-188 200 mg/kg caused even greater inhibition of tumor growth compared to radiation alone (p=0.006).

Thirty days after treatment, three mice that were given radiation+W-188 200 mg/kg (the optimal treatment in this experiment) were sacrificed and internal organs removed and processed for histopathological analysis by a board-certified pathologist at a commercial lab. All tissues examined were found to be normal including heart, lung, liver, spleen, kidney, stomach, and intestine.

These encouraging results suggest that non-toxic doses of W-188 can sensitize Bcl-2-overexpressing tumor cells to γ-radiation.

### Example 10

### W-143 Augments Dox Inhibition of Murine Tumor Cell Growth and Down Regulates Expression of Bcl-2 In Vivo

Since the previous in vivo studies assessed the activity of W-188, the present study was designed to evaluate the ability of W-143 to synergize with apoptosis inducers and inhibit tumor growth. W-143 was tested in the RIF-1 fibrosarcoma model. As described above, this model has been well-characterized, and naturally expresses high Bcl-2 levels and has a consistent growth pattern (Nahabedian, M. Y., et al., J. Natl. Can. Inst., 1988, 80:739; Chang, M-J., et al., Cancer Res., 1994, 54:5380; Srivastana, M., et al., J. Biol. Chem., 2001, 276:15481).

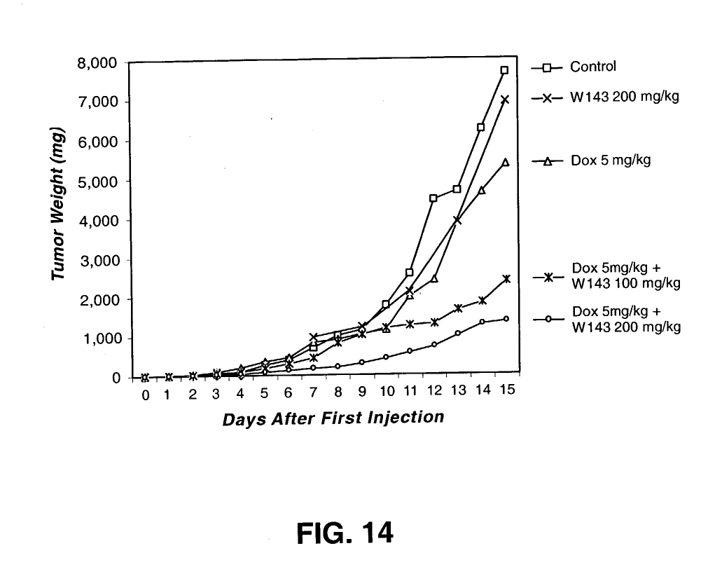

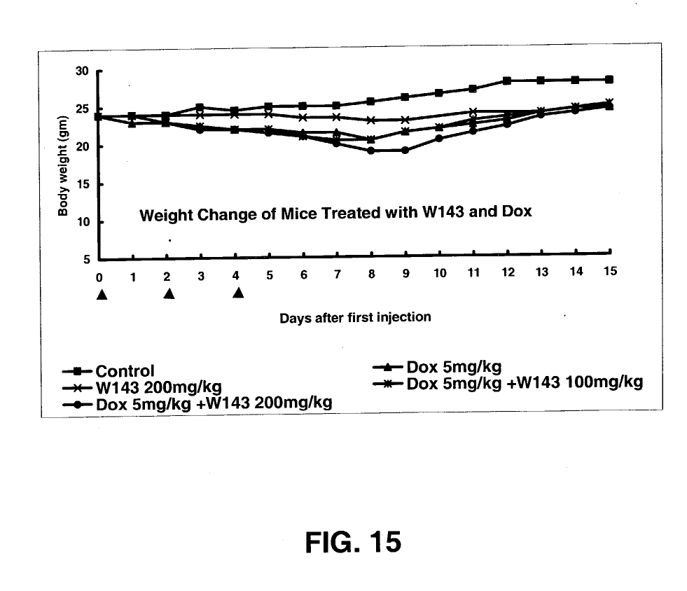

The protocol was similar to that described for the W-188 experiment in Example 8, except that in this study, mice with established tumors received only three injections of W-143 or Dox every other day starting on day 0. The results in FIG. 14 show that treatment with Dox 5 mg/kg alone or W-143 200 mg/kg alone had only a small effect on tumor growth. Treatment with W-143 100 mg/kg alone (data not shown) did not decrease tumor growth compared to the vehicle control. However, combined treatment with W-143 and Dox synergistically inhibited tumor growth. On day 15, Dox+W-143 200 mg/kg caused 83% inhibition of tumor growth (p=0.001), and Dox+W-143 100 mg/kg caused 70% inhibition (p=0.005) compared to vehicle control. The body weights of the mice in this model are shown in FIG. 15, which illustrates that Dox 5 mg/kg alone was toxic causing a significant weight loss on day 7-8. Dox combined with W-143 at 200 mg/kg was somewhat more toxic that Dox alone on day 8. However, these mice recovered, and weighed just as much as mice treated with Dox alone on day 13. Mice treated with Dox plus W-143 at 100 mg/kg did not show any greater weight loss than those treated with Dox alone at any time point. Mice treated with W-143 100 mg/kg alone weighed similar to the vehicle control mice (data not shown).

These findings demonstrate that a low non-toxic dose of W-143 will effectively synergize with Dox to inhibit RIF-1 tumor cell growth without causing significant side effects.

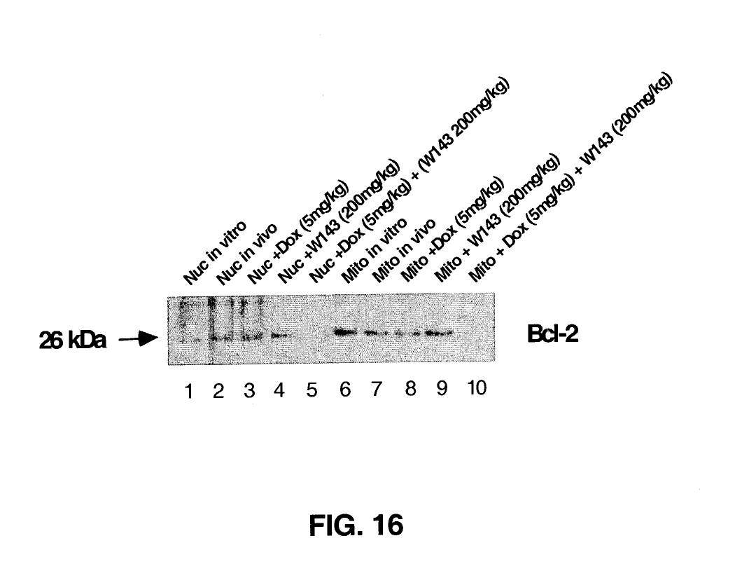

To examine the in vivo expression of Bcl-2, tumors were excised from mice on day 15 of the experiment shown in FIG. 14. Cells were dissociated, lysed, and the nuclei and mitochondria separated by differential centrifugation. RIF-1 cells cultured in vitro were processed in a similar manner as a positive control. Equal amounts of protein extracts were separated by electrophoresis and analyzed by Western blot for expression of Bcl-2. The results in FIG. 16 show that mitochondria (Mito) from cells grown in vitro or in vivo in untreated mice expressed high levels of Bcl-2. RIF-1 mitochondria from mice treated with Dox alone or W-143 alone also expressed high levels of Bcl-2. However, tumor cell mitochondria from mice treated with both Dox and W-143 had no detectable Bcl-2 (lane 10). The pattern of Bcl-2 expression was similar in the nuclei, although the overall levels were lower than in the mitochondria. There was no Bcl-2 detected in the cytosol in any of the samples (data not shown), indicating that the combined drug treatment downregulated expression of Bcl-2 as opposed to altering its subcellular distribution. This result came as a surprise, since preliminary data revealed that in vitro treatment of HL-60-Bcl-2 cells with W-188+chemotherapeutic drugs under conditions that sensitized the cells to apoptosis did not downregulate expression of Bcl-2. Most likely, this discrepancy can be explained by the fact that this line is transfected with exogenous Bcl-2, whereas the RIF-1 line expresses endogenous Bcl-2 under control of its natural promoter. If true, then this suggests that W-143 may have two different mechanisms of action: (1) downregulation of expression of natural endogenous Bcl-2; and (2) inhibition of the action of enforced overexpressed Bcl-2. Alternatively, W-188 and W-143 may differ in their mechanisms of action, although this conclusion seems less likely in view of the structural similarity of these two compounds.

TABLE 1
Curability of Cancers with Chemotherapy

| | |
|---|---|
| A. | Advanced cancers with possible cure |
| | Acute lymphoid and acute myeloid leukemia (pediatric/adult) |
| | Hodgkin's disease (pediatric/adult) |
| | Lymphomas—certain types (pediatric/adult) |
| | Germ cell neoplasms |
| | Embryonal carcinoma |
| | Teratocarcinoma |
| | Seminoma or dysgerminoma |
| | Choriocarcinoma |
| | Gestational trophoblastic neoplasia |
| | Pediatric neoplasms |
| | Wilm's tumor |
| | Embryonal rhabdomyocarcinoma |
| | Ewing's sarcoma |
| | Peripheral neuroepithelioma |
| | Neuroblastoma |
| | Small cell lung carcinoma |
| | Ovarian carcinoma |
| B. | Advanced cancers possibly cured by chemotherapy and radiation |
| | Squamous carcinoma (head and neck) |
| | Squamous carcinoma (anus) |
| | Breast carcinoma |
| | Carcinoma of the uterine cervix |
| | Non-small cell lung carcinoma (stage III) |
| | Small cell lung carcinoma |
| C. | Cancers possibly cured with chemotherapy as adjuvant to surgery |
| | Breast carcinoma |
| | Colorectal carcinoma ^{a} |
| | Osteogenic sarcoma |
| | Soft tissue sarcoma |
| D. | Cancers possibly cured with “high-dose” |
| | chemotherapy with stem cell support |
| | Relapsed leukemias, lymphoid and myeloid |
| | Relapsed lymphomas, Hodgkin's and non-Hodgkin's |
| | Chronic myeloid leukemia |
| | Multiple mycloma |
| E. | Cancers responsive with useful palliation, |
| | but not cure, by chemotherapy |
| | Bladder carcinoma |
| | Chronic myeloid leukemia |
| | Hairy cell leukemia |
| | Chronic lymphocytic leukemia |
| | Lymphoma—certain types |
| | Multiple myeloma |
| | Gastric carcinoma |
| | Cervix carcinoma |
| | Endometrial carcinoma |
| | Soft tissue sarcoma |
| | Head and neck cancer |
| | Adrenocortical carcinoma |
| | Islet-cell neoplasms |
| | Breast carcinoma |
| F. | Tumor poorly responsive in advanced stages to chemotherapy |
| | Pancreatic carcinoma |
| | Billiary-tract neoplasms |
| | Renal carcinoma |
| | Thyroid carcinoma |
| | Carcinoma of the vulva |
| | Colorectal carcinoma |
| | Non-small cell lung carcinoma |
| | Prostate carcinoma |
| | Melanoma |
| | Hepatocellular carcinoma |

^{a} Rectum also receives radiation therapy.

TABLE 2
Commonly Use Cancer ChemotherapyAgents

| Drug | Examples of Usual Doses |
|---|---|
| Alkylators | |
| Cyclophosphamide | 400-2000 mg/m² IV |
| | 100 mg/m² PO qd |
| Mechlorethamine | 6 mg/m² IV day 1 and day 8 |
| Chlorambucil | 1-3 mg/m² qd PO |
| Melphalan | 8 mg/m² qd × 5, PO |
| BCNU | 200 mg/m² IV |
| | 150 mg/m² PO |
| CCNU | 100-300 mg/m² PO |
| Ifosfamide | 1.2 g/m² per day qd × 5 + MESNA |
| Procarbazine | 100 mg/m² per day qd × 14 |
| DTIC | 375 mg/m² IV day 1 Nausea |
| | Flulike |
| Hexamethylmelamine | 260 mg/m² per day qd × 14-21 |
| | as 4 divided oral doses |
| Cisplatin | 20 mg/m² qd × 5 IV 1 q3-4 weeks |
| | or 100-200 mg/m²/dose IV q3-4 weeks |
| Carboplatin | 365 mg/m² IV q3-4 weeks as adjusted |
| | for CrCl |
| Antitumor antibiotics | |
| Bleomycin | 15-25 mg/d qd × 5 IV bolus or continuous IV |
| Actinomycin D | 10-15 μg/kg per day qd × 5 IV bolus |
| Mithramycin | 15-20 μg/kg qd × 4-7 (hypercalcemia) |
| | or 50 μg/kg qd × 3-8 (antineoplastic) |
| Mitomycin C | 6-10 mg/m² q6 weeks |
| Etoposide (VP16-213) | 100-150 mg/m² IV qd × 3-5d |
| | or 50 mg/m² PO qd × 21d |
| | or up to 1500 mg/m²/of dose (high |
| | dose with stem cell support) |
| Teniposide (VM-26) | 150-200 mg/m² twice per week for 4 weeks |
| Amsacrine | 100-150 mg/m² IV qd × 5 |
| Topotecan | 20 mg/m² IV q3-4 weeks over 30 min |
| | or 1.5-3 mg/m² q3-4 weeks over 24 h |
| | or 0.5 mg/m² per day over 21 days |
| Irinotecan (CPT II) | 100-150 mg/m² IV over 90 min q3-4 weeks |
| | or 30 mg/m² per day over 120 h |
| Doxorubicin and | 45-60 mg/m² dose q3-4 weeks |
| daunorubicin | or 10-30 mg/m² dose q week |
| | or continuous-infusion regimen |
| Idarubicin | 10-15 mg/m² IV q3 weeks |
| | or 10 mg/m² IV qd × 3 |
| Epirubicin | 150 mg/m² IV q3 weeks |
| Mitoxantrone | 12 mg/m² qd × 3 |
| | or 12-14 mg/m² q3 weeks |
| Antimetabolites | |
| Deoxycoformycin | 4 mg/m² IV every other week |
| 6-Mercaptopurine | 75 mg/m² PO |
| | or up 500 mg/m² PO (high dose) |
| 6-Thioguanine | 2-3 mg/kg per day for up to 3-4 weeks |
| Azathioprine | 1-5 mg/kg per day |
| 2-Chlorodeoxyadenosine | 0.09 mg/kg per day qd × 7 as continuous |
| | infusion |
| Hydroxyurea | 20-50 mg/kg (lean body weight) PO qd |
| | or 1-3 g/d |
| Methotrexate | 15-30 mg PO or IM qd × 3-5 |
| | or 30 mg IV days 1 and 8 |
| | or 1.5-12 g/m² per day (with leucovorin) |
| 5-Fluorouracil | 375 mg/m² IV qd × 5 |
| | or 600 mg/m² IV days 1 and 8 |
| Cytosine | 100 mg/m² per day qd × 7 by continuous |
| arabinoside | infusion or 1-3 g/m² dose IV bolus |
| Azacytidine | 750 mg/m² per week |
| | or 150-200 mg/m² per day × 5-10 (bolus) or |
| | (continuous IV) |
| Gemcitabine | 1000 mg/m² IV weekly × 7 |
| Fludarabine | 25 mg/m² IV qd × 5 |
| phosphate | |
| Asparaginase | 25,000 IU/m² q3-4 weeks |
| | or 6000 IU/m² per day qod for 3-4 weeks |
| | or 1000-2000 IU/m² for 10-20 days |
| Antimitotic agents | |
| Vincristine | 1-1.4 mg/m² per week |
| Vinblastine | 6-8 mg/m² per week |
| Vinorelbine | 15-30 mg/m² per week |
| Paclitaxel | 135-175 mg/m² per 24-h infusion |
| | or 175 mg/m² per 3-h infusion |
| | or 140 mg/m² per 96-h infusion |
| | or 250 mg/m² per 24-h infusion plus G-CSF |
| Docetaxel | 100 mg/m² per 1-h infusion q3 weeks |
| Estramustine | 14 mg/kg per day in 3-4 divided doses with |
| phosphate | water >2 h after meals; Avoid Ca²⁺ -rich foods |

TABLE 3
W-188 does not enhance chemotherapeutic
drug-induced cytotoxicity of normal venous endothelial cells*

| Drug | % cytotoxicity |
|---|---|
| Etoposide (0 μM) + W-188 (50 μg/mL) | 22 ± 2.0 |
| Etoposide (0 μM) + W-188 (25 μg/mL) | 7 ± 1.5 |
| Etoposide (100 μM) + W-188 (0 μg/mL) | 22 ± 3.4 |
| Etoposide (100 μM) + W-188 (50 μg/mL) | 21 ± 1.6 |
| Etoposide (100 μM) + W-188 (25 μg/mL) | 11 ± 1.5 |
| YW-200 (0.01 μM) + W-188 (0 μg/mL) | 57 ± 5.1 |
| YW-200 (0.01 μM) + W-188 (50 μg/mL) | 59 ± 2.2 |
| YW-200 (0.01 μM) + W-188 (25 μg/mL) | 38 ± 4.9 |

*Normal human venous endothelial cells were incubated with the indicated drugs for a total of 6 h and cell death was determined using the MTT dye reduction assay which measures the production of formazan from the substrate 3-(4,5-dimethylthiazol-2-yl)-2,5-diphenyltetrazolium bromide (MTT).

TABLE 4
Evaluation of W-188 analogs for Bcl-2-sensitizing activity

| Exp | | | % DNA |
|---|---|---|---|
| # | Cell Line | Cell Treatment | Fragmentation |
| 1 | HL-60 neo | etoposide 5 μM | 72 |
| | HL-60 Bcl-2 | etoposide 5 μM | 0 |
| | HL-60 Bcl-2 | etoposide 5 μM + W-188 10 μM | 76 |
| | HL-60 Bcl-2 | etoposide 5 μM + W-188 5 μM | 40 |
| | HL-60 Bcl-2 | etoposide 5 μM + 4,5-dicyanoimidazole 10 μM | 10 |
| | HL-60 Bcl-2 | etoposide 5 μM + cobalt (II) pthalocyanine 10 μM | 9 |
| | HL-60 Bcl-2 | etoposide 5 μM + 2,2′-bis(4,5-dimethyl-imidazole) 10 μ | 2 |
| | HL-60 Bcl-2 | etoposide 5 μM + 1,4,8,11-tetraazacyclotetra-decane-1,4,8,11-tetraacetic acid | 0 |
| | | tetrahydrochioride hydrate 10 μM | |
| | HL-60 Bcl-2 | etoposide 5 μM + deuterporphyrin IX dimethyl ester 10 μM | 8 |
| | HL-60 Bcl-2 | etoposide 5 μM + hematin 10 μM | 2 |
| | HL-60 Bcl-2 | etoposide 5 μM + 4-cyano tempo 10 μM | 2 |
| 2 | HL-60 neo | etoposide 10 μM | 57 |
| | HL-60 Bcl-2 | etoposide 10 μM | 0 |
| | HL-60 Bcl-2 | etoposide 10 μM + W-188 10 μM | 62 |
| | HL-60 Bcl-2 | etoposide 10 μM + porphyin 10 μM | 10 |
| | HL-60 Bcl-2 | etoposide 10 μM + coproporphyrin 10 μM | 15 |
| | HL-60 Bcl-2 | etoposide 10 μM + bilirubin 10 μM | 17 |
| | HL-60 Bcl-2 | etoposide 10 μM + protoporphyrin IX 10 μM | 3 |
| 3 | HL-60 neo | etoposide 5 μM | 97 |
| | HL-60 Bcl-2 | etoposide 5 μM | 24 |
| | HL-60 Bcl-2 | etoposide 5 μM + W-188 10 μM | 92 |
| | HL-60 Bcl-2 | etoposide 5 μM + biliverdin 20 μM | 20 |
| | HL-60 Bcl-2 | etoposide 5 μM + Zn protoporphyrin IX 20 μM | 19 |
| | HL-60 Bcl-2 | etoposide 5 μM + Sn protoporphyrin IX 20 μM | 5 |
| | HL-60 Bcl-2 | etoposide 5 μM + Fe protoporphyrin IX 20 μM | 17 |
| 4 | HL-60 neo | etoposide 5 μM | 63 |
| | HL-60 Bcl-2 | etoposide 5 μM | 3 |
| | HL-60 Bcl-2 | etoposide 5 μM + W-188 10 μM | 59 |
| | HL-60 Bcl-2 | etoposide 5 μM + 2,9,16,23-tetra-tert-butyl-29H, 31H-phthalocyanine 10μM | 0 |
| | HL-60 Bcl-2 | etoposide 5 μM +2,3,7,8,12,13,17,18-octaethyl 21H, 23H-porphine iron (III) acetate | 3 |
| | | 10 μM | |
| | HL-60 Bcl-2 | etoposide 5 μM +5,10,15,20-tetra-p-tolyl-21H,23H-porphine 10 μM | 15 |
| | HL-60 Bcl-2 | etoposide 5 μM +etioporphyrin I dihydrobromide 10 μM | 25 |
| | HL-60 Bcl-2 | etoposide 5 μM +5,10,15,20-tetrakis (pentafluorophenyl)-21H,23H-porphine 10 μM | 22 |
| 5 | HL-60 neo | etoposide 10 μM | 57 |
| | HL-60 Bcl-2 | etoposide 10 μM | 0 |
| | HL-60 Bcl-2 | etoposide 10 μM + W-188 10 μM | 55 |
| | HL-60 Bcl-2 | etoposide 10 μM + W-125 10 μM | 26 |
| | HL-60 Bcl-2 | etoposide 10 μM + W-135 10 μM | 22 |
| | HL-60 Bcl-2 | etoposide 10 μM + W-137 10 μM | 28 |
| | HL-60 Bcl-2 | etoposide 10 μM + W-141 10 μM | 32 |
| | HL-60 Bcl-2 | etoposide 10 μM + W-143 10 μM | 34 |
| | HL-60 Bcl-2 | etoposide 10 μM + W-145 10 μM | 28 |

TABLE 5
W-188 Sensitizes Bcl-2-Overexpressing Cells to
Etoposide-Induced Activation of AP24

| | | AP24 Activity |
|---|---|---|
| Cell Line | Drug | (AAPVase, nmol/mg/hr) |
| HL-60 neo | — | 10 |
| HL-60 neo | Etoposide 50 μM | 100 |
| HL-60 Bcl-2 | — | 11 |
| HL-60 BcI-2 | Etoposide 50 μM | 14 |
| HL-60 Bcl-2 | Etoposide 50 μM + W-188 | 124 |
| | 5 μg/ml | |
| PW neo | — | 11 |
| PW neo | Etoposide 50 μM | 88 |
| PW Bcl-2 | — | 9 |
| PW Bcl-2 | Etoposide 50 μM | 12 |
| PW Bcl-2 | Etoposide 50 μM + W-188 | 129 |
| | 5 μg/ml | |

HL-60 and PW leukemia lines transfected with Bcl-2 or vector control were cultured with and without etoposide ± W-188 for 3 h, and AP24 was purified from the cytosol by affinity chromatography and assayed on the Ala-Ala-Pro-Val-pNa (AAPVase) synthetic substrate as described in detail previously (Wright, S. C., et al., J. Exp. Med., 1994, 180:2113).

TABLE 6
W-188 Sensitizes Nuclei from Bcl-2 Overexpressing
Cells to DNA Fragmentation Induced by AP24

| | ± W-188 | | |
|---|---|---|---|
| Nuclei | Treatment | Dilution AP24 | % DNA Fragmentation |
| HL-60 neo | − | 1:10 | 33 ± 0.4 |
| HL-60 neo | − | 1:20 | 24 ± 1.0 |
| HL-60 Bcl-2 | − | 1:10 | 0 |
| HL-60 Bcl-2 | − | 1:20 | 0 |
| HL-60 Bcl-2 | + | 1:10 | 44 ± 4.8 |
| HL-60 Bcl-2 | + | 1:20 | 19 ± 1.0 |

Cells were incubated ± W-188 5 μg/ml for 4 hr and then nuclei were prepared and tested for sensitivity to AP24-induced DNA fragmentation in a 20 hr assay as described in detail previously (Wright, S. C., et al., Cancer Res., 1998, 58:5570).

TABLE 7
Subcellular Distribution of W-188

| | | W-188 (μg/mg protein) | |
|---|---|---|---|
| Cell Line | Cytosol | Mitochondria | Nuclei |
| HL-60 neo | 0 | 3.3 | 100 |
| HL-60 Bcl-2 | 0.25 | 10.0 | 186 |
| PW neo | 0 | 2.5 | 133 |
| PW Bcl-2 | 0 | 4.5 | 166 |

### Equivalents

From the foregoing detailed description of the specific embodiments of the invention, it should be apparent that unique compositions and a unique procedure to treat apoptosis-resistant cells has been described resulting in reversing the resistance and increasing the sensitivity of apoptosis-resistant cells to apoptosis inducers. Although particular embodiments have been disclosed herein in detail, this has been done by way of example for purposes of illustration only, and is not intended to be limiting with respect to the scope of the appended claims that follows. In particular, it is contemplated by the inventor that substitutions, alterations, and modifications may be made to the invention without departing from the spirit and scope of the invention as defined by the claims. For instance, the choice of apoptosis inducers, or the choice of doses, or the choice of route of administration of the compositions of the present invention is believed to be matter of routine for a person of ordinary skill in the art with knowledge of the embodiments described herein.

We claim:

1. A method of reversing the resistance of an apoptosis-resistant cell, the method comprising administering a compound selected from the group of compounds consisting of the formula: 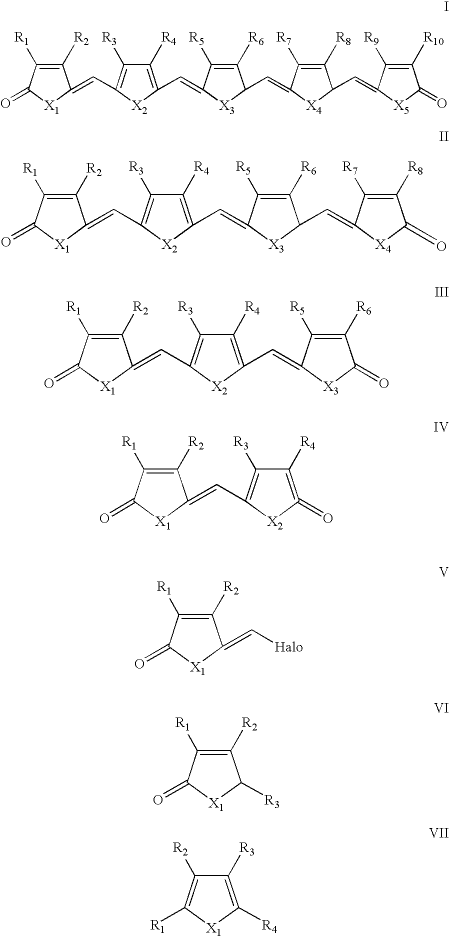
wherein:
R₁, R₂, R₃, R₄, R₅, R₆, R₇, R₈, R₉, and R₁₀ are independently H, CH═CH₂, C₁-C₅ alkyl, C₁-C₄ alkyl-COOH, and C₁-C₄ alkyl-COOCH₃, and wherein R₁, R₂, R₃, R₄, R₅, R₆, R₇, R₈, R₉, and R₁₀ can be the same or different;
X₁, X₂, X₃, X₄, and X₅ are independently N, O or S, and wherein X₁, X₂, X₃, X₄, and X₅ can be the same or different; and
Halo is F, Cl, Br or I, and
wherein the compound increases the sensitivity of the apoptosis-resistant cell to an apoptosis inducer.

2. The method of claim 1, wherein the compound is selected from the group consisting of:

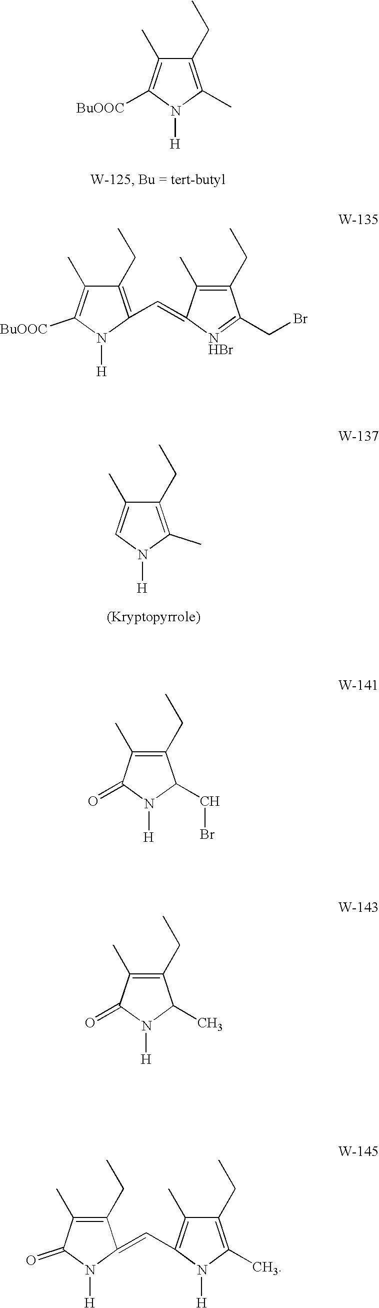

3. The method of claim 1, wherein the compound has the formula:

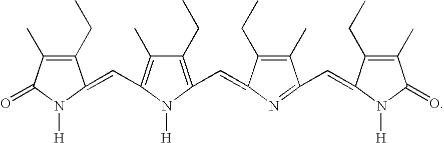

4. The method of claim 1, wherein the compound has the formula:

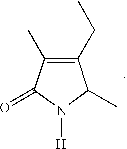

5. The method of claim 1, wherein the compound is administered simultaneously or sequentially with at least one apoptosis inducer.

6. The method of claim 5, wherein the apoptosis inducer is an apoptosis-inducing cancer therapy selected from the group consisting of: radiation therapy, chemotherapy, and biologic therapy.

7. The method of claim 6, wherein the chemotherapy is selected from the group consisting of: an antimetabolite, an alkylating agent, a plant alkaloid, and an antibiotic.

8. The method of claim 7, wherein the antimetabolite is selected from the group consisting of: methotrexate, 5-fluoruracil, 6-mercaptopurine, cytosine arabinoside, hydroxyurea, and 20chlorodeoxy adenosine.

9. The method of claim 7, wherein the alkylating agent is selected from the group consisting of: cyclophosphamide, melphalan, busulfan, cisplatin, paraplatin, chlorambucil, and nitrogen mustards.

10. The method of claim 7, wherein the plant alkaloid is selected from the group consisting of: vincristine, vinblastine, and VP-16.

11. The method of claim 6, wherein the antibiotic is selected from the group consisting of: doxorubicin, daunorubicin, mitomycin c, and bleomycin.

12. The method of claim 6, wherein the chemotherapy is selected from the group consisting of: decarbazine, mAMSA, hexamethyl melamine, mitroxantrone, taxol, etoposide, dexamethasone.

13. The method of claim 6, wherein the radiation therapy is selected from the group consisting of: photodynamic therapy, radionucleotides, and radioimmunotherapy.

14. The method of claim 6, wherein the biologic therapy is selected from the group consisting of: immunotherapy, differentiating agents, and agents targeting cancer cell biology.

15. The method of claim 1, wherein the apoptosis-resistant cell overexpresses Bcl-2.

16. The method of claim 1, wherein the apoptosis-resistant cell overexpresses Bcl-X_{L}.
